(19) United States
(12) Patent Application Publication
(10) Pub. No.: US 2010/0233111 A1
(43) Pub. Date: Sep. 16, 2010

(54) GASTROPOD BIOLOGICAL FLUID, METHOD OF MAKING AND REFINING AND USE

(76) Inventors: William Wang, Iowa City, IA (US); Jun Yi, Wuhan City (CN); Sheng Ke, Wuhan City (CN); Maria Halmela, Laitila (FI)

Correspondence Address: MCANDREWS HELD & MALLOY, LTD, 500 WEST MADISON STREET, SUITE 3400, CHICAGO, IL, 60661, US

(21) Appl. No.: 12/643,751

(22) Filed: Dec. 21, 2009

Related U.S. Application Data
(63) Continuation of application No. PCT/US2008/068047, filed on Jun. 24, 2008.
(60) Provisional application No. 61/123,313, filed on Apr. 6, 2008.

(51) Int. Cl. A61K 8/18; A61K 31/715; A61K 38/43; A61K 38/46; A61Q 19/02; A61K 36/54; A61K 36/752; A61K 36/00

(52) U.S. Cl. 424/62; 514/54; 424/94.1; 424/94.6; 424/739; 424/736; 424/774; 424/773

(57) ABSTRACT

The present technology generally relates to skin care compositions, cosmeceuticals or formulations and methods of making or using the same. More specifically, the presently described technology generally relates to methods of making and using compositions, cosmeceuticals or formulations including a unique composition collected and refine from a gastropod, namely Helix Aspersa Müller, among others.

### RELATED APPLICATIONS

The present application is a continuation of PCT Application No. PCT/US2008/068047 with an International Filing Date of Jun. 24, 2008, which relates to and claims the benefit of U.S. Provisional Application No. 60/937,008 filed on Jun. 23, 2007 entitled BIOMEMBRANE PRODUCTS FOR SKIN RENEWAL, WOUND REPAIR AND SCAR REMOVAL and Provisional Application filed on Apr. 6, 2008, with Express Mail Label No. EV 332 942 220 US, Attorney Docket No. 40064PIIA entitled BIOCREAM PRODUCTS FOR SKIN RENEWAL, WOUND REPAIR, AND SCAR REMOVAL. The foregoing applications are herein incorporated by reference in their entirety.

### BACKGROUND OF THE INVENTION

The present technology generally relates to skin care compositions, cosmeceutical compositions and methods of making or using the same. More specifically, the presently described technology generally relates to skin care compositions, cosmocuetical compositions as well as methods of making and using the same utilizing a unique composition recovered and refine from a gastropod, namely Helix Aspersa Müller, among others.

Skin is the largest organ of the body system made up of multiple layers of epithelial tissues that guard underlying muscles and organs. Because it interfaces with the environment, skin plays a vital role in protecting the body against pathogens and other environmental harms. Skin also plays an important role in other functions such as insulating, temperature regulation, sensation, synthesis of vitamin D, and the protection of vitamin B folates. Therefore, its imperative to maintain healthy skin and promote healing to damaged or wounded skin. Many conventional skin care products on the market today contain for example chemically synthesized ingredients that do not always help the skin heal, rather may cause detrimental side effects.

Unlike many skin care products, the present technology is based in traditional Chinese medicine where the active ingredients that promote skin repair and healing are derived and refined from natural sources. Glycoconjugates are the general classification for carbohydrates covalently linked with other chemical species and have been found to be very important compounds in biology and consist of many different categories such as glycoproteins, glycopeptides, peptidoglycans, glycolipids, and lipopolysaccharides. Glycoconjugates are involved in cell-cell interactions, including cell-cell recognition, and cell-matrix interactions. Interestingly, there has been renewed interest into the benefits of unique glycoconjugates excreted by gastropods that may provide medicinal benefits.

Gastropods, or snails, are a diversified class belonging to the phylum of mollusks, with 60,000-75,000 known living species. Snails have been known to provide medicinal benefits for centuries, but it as not been until recently that a better understanding of the makeup of snails, snail body parts and/or snail secretions has been shown to have beneficial properties. Pursuant to the present technology described herein, it has been surprisingly found that there is a unique composition of collected and refined biological fluid excreted by gastropods under stress that provides skin healing and rejuvenating properties. This excreted biological fluid may be used in many skin care compositions and other formulations for the treatment a number of skin ailments, for example wounds, burns, scars, keratosis, psoriasis, acne, wrinkles and age and skin damage.

### BRIEF SUMMARY OF THE INVENTION

In one aspect, the present technology provides a unique method of collecting and refining a gastropod biological fluid from a live gastropod. The method includes, for example, the steps of stimulating the gastropod to increase the secretion of the desired biological fluid. Methods of stimulating the gastropod include, for example, exposing the gastropod to sound vibration; exposing the gastropod to low hyperbaric pressure; exposing the gastropod to thermal puncture; exposing the gastropod to a physical stimulus; exposing the gastropod to ionizing radiation; exposing the gastropod to gravity; or inverting the gastropod. The method further includes the step of collecting the secreted fluid and centrifuging the secreted fluid to create a supernatant. The method also includes the step of filtering the supernatant to recover a filtrate containing an enhanced amount of glycosaminoglycans, a type of glycoconjugate. It should be appreciated that the present technology includes the gastropod biological fluid collected from the described methodology.

In another aspect, the present technology provides one or more cosmeceuticals, including an effective amount of the collected and refined gastropod biological fluid. The recovered and refined gastropod biological fluid (e.g. the filtrate or supernatant) may be about 1% to about 30% by weight of the compositions described herein. Alternatively, the gastropod biological fluid may include about 10% to about 20% of the one or more skin care compositions, formulations or cosmeceutical described herein. The compositions may be used for skin repair and rejuvenation, treatment of wounds, acne, and scars, and/or may be used as a sunscreen, an anti-aging cream, an anti-wrinkle cream, a moisturizing cream, or as a whitening cream. The presently described compositions may further include an effective amount of squalane and an effective amount of hyaluronic acid, among other additives.

In a further aspect, the present technology provides a biologically effective amount of a gastropod excreted fluid and a delivery system capable of being applied to the skin. Delivery systems may, for example, include creams, gels, solutions, oils, suspensions, dispersions, ointments, membranes, patches, powders, suppositories, capsules, pills, soaps and skin adhesive glues.

In a still further aspect of the presently described technology, there is provided a cosmeceutical, composition or formulation that promotes skin rejuvenation and healing comprising at least about 15% by weight of a gastropod biological filtrate.

In another aspect, the present technology provides a gastropod biological fluid including about 0.002% to about 0.1% uronic acid; about 0.003% to about 0.5% hexozamine; about 0.001 to about 0.1% acetyl; about 0.001 to about 0.1% sulfate; about 0.02% to about 1.0% of at least one mucopolysaccharide; about 0.05% to about 0.5% of at least one fibroblast growth factor; about 0.01% to about 0.1% of at least one enzyme; about 0.01% to about 1.0% hyaluronic acid; about 0.001% to about 0.2% of at least one oxygen carrier; about 0.005% to about 0.1% of at least one high molecular weight protein; about 0.0001 to about 0.01% of at least one trace element; about 0.005% to about 0.1% of at least one high molecular weight antioxidant compound; about 0.005% to about 0.1% of at least one low molecular weight compound; and/or about 80% to about 98% water.

In an alternative aspect, the present technology provides a composition, cosmeceutical or formulation having at least about 1% to at least about 30% of a gastropod biological fluid. The gastropod biological fluid may be a supernatant or a filtrate containing enriched amounts of glycosaminoglycans.

In yet another aspect, the present technology provides a composition, cosmeceutical, or formulation having about 10.0% to about to about 25.0% of a gastropod excreted fluid. The composition, cosmeceutical, or formulation further include about 0.5% to about 10.0% stearic acid; about 0.1% to about 1.0% single stearic acid glyceride; about 1.0% to about 10.0% of at least one oil; about 0.1% to about 1.0% octadecanol; about 1.0% to about 10.0% glycerin; about 0.02% to about 2.0% potassium hydroxide; and/or about 0.2% to about 2.0% of a preservative.

In a further aspect, the present technology provides a composition, cosmeceutical, or formulation having about 10% to about to about 20% of a gastropod excreted fluid; about 0.2% to about to about 2.0% rose hip seed oil; about 0.2% to about 2.0% of a olive oil emulsifier; about 0.5% to about 5.0% of a seaweed extract; about 0.1% to about 1.0% of hyaluronic acid; about 0.4% to about 4.0% chamomile extract; about 0.5% to about 5.0% saccharide isomerate; about 0.3% to about 3.0% licorice extract; and/or about 0.3% to about 3.0% of a preservative.

### BRIEF DESCRIPTION OF SEVERAL VIEWS OF THE DRAWINGS

FIG. 1 is a picture of an SDS page gel showing the digestion of the gastropod biological fluid GAG with heparin lyase I, heparin lyase II, or heparin lyase III in comparison to a control.

FIG. 2 is a set of pictures depicting histological staining of the mantle of Helix Aspersa Müller. FIG. 1-2 depicts WPA staining; FIG. 2-2 depicts nNOS staining; FIG. 2-3 depicts staining of proteoglycans with carboxyl groups; FIG. 2-4 depicts carboxylic acid proteoglycan staining; FIG. 2-5 depicts other glycoproteins staining; FIG. 2-6 depicts -D-glucose and δ-D-mannose staining; FIG. 2-7 depicts oligosaccharides staining; FIG. 2-8 depicts δ-D-gal and (1-3)-D-Gal-Nac staining; FIG. 2-9 depicts δ-L-fucose staining; and FIG. 2-10 depicts N-acetyl-D-galactosamine or D-galactose staining.

FIG. 3 is a line graph depicting the increase in cell proliferation of fibroblasts treated with gastropod biological fluid (snail serum, SJ) in the presence and absence of citrate.

FIG. 4 illustrates histological staining of fibronectin assembly in CHO-K1 cells treated with gastropod biological fluid (SJ) of the present technology.

FIG. 5 illustrate histological staining of human dermal fibroblast morphology after treatment with gastropod biological fluid of the present invention.

FIG. 6 is a line graph depicting the antioxidant qualities of gastropod biological fluid (Snail serum).

FIG. 7 is a line graph depicting the moisturizing qualities of formulations of a moisturizing liquid containing gastropod biological fluid.

FIG. 8 is a graph depicting the long term (22 day) moisturizing effect of formulation containing gastropod biological fluid on healthy test subject's skin.

FIG. 9 are three dimensional graphs showing the effects of anti-wrinkle cream on wrinkle depths for groups treated with a control cream (A, B, C) and an anti-wrinkle cream containing gastropod biological fluid of the present technology (D, E, F). Measurements were taken at 0 days treatment (A, D), 15 days treatment (B, E) or 30 days treatment (C, F).

As noted herein, the present technology relates generally to a unique method collecting and refining a gastropod biological fluid from live gastropods and the use of such gastropod biological fluid in compositions, cosmeceuticals, or formulations that provide, for example, beneficial skin care treatments and therapeutic outcomes. The naturally-based compositions, cosmeceuticals, and/or formulations may trigger the regeneration of damaged cells; prevent or reduce scarring, keratosis, psoriasis scales, as well as all types of skin blemishes; prevent or reduce the appearance of acne; prevent or reduce the signs of wrinkles; and can replenish and/or moisturize the lipid barrier of the skin. The described compositions, cosmeceuticals, and/or formulations are based on natural ingredients containing preferably little or no chemicals, detergents or fillers. In doing so, it is believed (although not wanting to be bound by any particular theory) that the unique compositions having such natural ingredients provide antimicrobial, protective, moisturizing, repairing, and/or renewing properties to the skin, including components or component systems that mimic the intracellular structure of skin and are essential for normal skin function.

In one embodiment, the present technology provides a method of collecting and refining a gastropod biological fluid from a live gastropod. At least one advantage of the present technology is the ability to use the gastropod repeatedly for collection of the biological fluid since the process preferably does not harm the gastropod. Gastropods secrete the biological fluid to be collected and refined when under stress in order to protect, moisturize, repair and renew its own skin membrane. The method comprises the steps of stimulating a live gastropod to increase biological fluid secretion; collecting the secreted fluid; centrifuging the secreted fluid to create a supernatant; and filtering the supernatant to recover a filtrate. This filtrate contains an enhanced amount of one or more unique gastropod glycosaminoglycans.

As used herein, a gastropod is a member of the Phylum Mollusca class Gastropoda, including both Helix (shelled) and non-Helix (shell-less) members whose glandular secreted fluid is used as the active ingredient in the practice of the present technology. Suitable gastropods include, for example, Helix pomatia, Helix hortensis, Helix nemoralis, Helix cardidula, Helix tchthyomma (also referred to as Helix campylea), Helix fructicicola (also referred to as Helix se uca), Helix strigella, Helix fruticum, Helix bidens, Helix arbostorum, Helix rotundata, Helix aculeata, Helix pulchella, Helix personata, Helix holoserica, Helix aperta, Helix parnassia, Helix alonensis, Helix candidissima, Helix pisana, and Helix gualteviana. A more extensive discussion of gastropoda may be found in Invertebrate Zoology (5th Ed.), 864 p. Robert D. Barnes, (Saunders College Publishing, Inc., Troy, Mich., 1987). A particularly suitable gastropod is Helix Aspersa Müller. Alternatively, another suitable gastropod is Helix pomatia.

At least one method of the presently described technology comprises stimulating a live gastropod to increase biological fluid secretion. Methods to stimulate a live gastropod may include any methods that do not harm the live gastropod while increasing the biological fluid output of the gastropod, allowing the gastropod to be re-used for biological fluid collection. Stimulation causes the gastropod to increase biological fluid secretions naturally produced by the mucinous, albuminous, and salivary glands. These methods include, but are not limited to, exposing the gastropod to sound vibrations, exposing the gastropod to low hyperbaric pressure, exposing the gastropod to thermal punctures, exposing the gastropod to hypoxic conditions, exposing the gastropod to gravitation force, inverting the gastropod to alter its orientation, and exposing the gastropod to a physical stimulus.

Exposure of a gastropod to sound vibrations includes methods known in art to expose the gastropod to frequencies in the range of about 5 kHz, to about 25 kHz, alternatively about 10 KHz to about 20 kHz, alternatively between about 15 kHz to about 20 kHz for about 1 second to about 60 seconds, alternatively between about 5 seconds to about 30 seconds, more suitably about 10 seconds. The gastropod may be exposed to the sound frequencies of about 5 kHz, about 10 kHz, about 12 kHz, about 15 kHz, about 20 kHz, for a period of at least about 5 seconds, at least about 10 seconds, at least about 15 seconds, at least about 20 seconds to increase secretion of the biological fluid.

A particularly suitable method of exposing a gastropod to sound vibrations includes exposing the gastropod to ultrasonic irradiation by an ultrasonic generator UP 200H horn type (20 kHz with maximum wave amplitude of 210 μm and a maximum nominal power output of 460W) equipped with a radial Sonotrode S3 (3 mm diameter) for about 10 seconds.

Alternatively, the gastropod may be stimulated to increase production of the biological fluid to be collected and refined by exposing the gastropod to low hyperbaric pressure. Any suitable means known in the art may be used including hyperbaric oxygen therapy (HBOT) chambers. Suitable HBOT chambers may expose the gastropod to pressures of about 3 pounds per square inch (psi) to about 8 psi, more suitably about 4 psi (1.27 ATA (atmospheres absolute) 8.92 FSW) to about 7.35 psi (1.5 ATA 16.38 FSW) with an oxygen concentration of at least about 90%, more suitably at least about 95%, more suitably at least about 100%. The gastropod is exposed to the low hyperbaric pressure for at least about 5 seconds, more suitable at least about 10 seconds, more suitably at least about 12 seconds, more suitably at least about 15 seconds to stimulation the gastropod to produce the biological fluid.

Stimulating a gastropod to increase biological fluid secretion may also include inverting a gastropod for a specified amount of time. A gastropod may be inverted by hanging the gastropod by its tail with its head toward the ground for at least about 5 seconds, at least about 7 seconds, at least about 10 seconds, at least about 30 seconds, at least about 1 minute, at least about 1.5 minutes, at least about 2 minutes. For example, minipliers may be used to clip to the tail of the gastropod and invert the snail in the air for about 10 seconds to about 2 minutes. A straw may be used to collect the biological fluid along the ventral surface of the gastropod.

Alternatively, stimulating a gastropod to increase biological fluid secretion may include exposing the gastropod to gravity. The gravitational force causes an increase in biological fluid secretion in the sinus region of the gastropod, such as the buccal or cardiac sinus. The biological fluid may be extracted using a syringe to remove the biological liquid.

To increase fluid secretion, gastropods may also be exposed to ionizing radiation, either x-rays or gamma rays, for about 0.5 seconds, for about 1.0 seconds, for about 2.0 seconds. Gastropods may also be exposed to hypoxic conditions to stimulate fluid secretion. The gastropod is exposed to oxygen depletion or environmental hypoxia for at least about 10 seconds, at least about 12 seconds, at least about 15 seconds to increase production of secretion. Any method of oxygen depletion or environmental hypoxia known by one familiar in the art may be used.

In another embodiment, stimulating a gastropod to increase biological fluid secretion can be done, for example, by exposing the gastropod to physical stimulation. A suitable means of physically stimulating a gastropod is centrifuging the gastropod. Centrifuging is carried out under conditions which will not break the shell of the shelled gastropod or if a shell-less gastropod, under conditions in which the gastropod remains alive and is not crushed. The gastropod is centrifuged for a period of time at a temperature and gravity (G) sufficient to cause the gastropod to secrete fluids. The force required to stimulate fluid production in a gastropod is suitable less than about 7G, more suitable less than about 6 G, more suitably between about 1 G and about 6 G, more suitably between about 2 G and about 5 G for about 2 minutes to about 10 minutes. One suitable embodiment includes, for example, centrifuging the gastropod at or about 2 G for about 10 minutes. The gastropod is centrifuged at temperatures of about 10° C. or greater, more suitably 15° C. or greater, more suitably 20° C. or greater, more suitably between about 10° C. and about 35° C., more suitably between about 20° C. and about 35° C.

An alternative method of centrifugation includes pulsating the gastropod for about 3 to about 4 pulsations during the centrifugation. A pulsation includes accelerating the centrifuge to the desired G force, decelerating the centrifuge and accelerating the centrifuge again. A suitable embodiment of this method includes centrifuging the gastropod at about 2 G for a period of about 10 minutes with about 3 or about 4 pulsations being performed during this time period.

The method of collecting a gastropod biological fluid after stimulating the gastropod may be by any means known to one familiar in the art. For example, a straw may be used to siphon the biological fluid from the ventral surface of the gastropod.

The collected biological gastropod secreted fluid may be centrifuged at about 200 rpm to about 5000 rpm, more suitably about 2000 rpm in a for at least about 2 minutes, alternatively at least about 5 minutes, alternatively at least about 2 minutes to about 10 minutes to remove any large particles and to produce a supernatant. The supernatant is decanted and filtered through a microporous filter having a pore size of about 0.1 to about 1 micron to produce a filtrate. Filtration may be carried out under pressure to speed up the filtration process. The filtrate containing the active ingredients in the gastropod biological fluid is then recovered.

In some embodiments, the method of collecting a biological fluid from a live gastropod further includes fasting the gastropod for at least about 1 day to about 5 days, alternatively about 1 day to about 2 days before stimulation of the gastropod. Again, not wanting to be bound by any particular theory, it is believed that fasting of the gastropod decreases the amount of toxins that may be present in the secreted fluids produced as a byproduct of the food the gastropod eats. Alternatively, if the gastropod is not fasted, the toxins may be removed from the secreted fluid by methods known by those familiar in the art to inactivate the toxins, for example

The present technology further provides the gastropod biological fluid including a supernatant and a filtrate made by the process described above. As described in the examples below, the gastropod biological fluid contains a mixture of components that among other properties provide beneficial healing and rejuvenating effects to human skin. When applied to the skin using a suitable delivery vehicle, the gastropod biological fluid may, for example, promote the immune system to clear our dead and dying cells, trigger the production of new cells, rebuilds the skin matrix by repairing, moisturizing, and protecting dermal elements. The gastropod biological fluid includes about 0.001% to about 0.1% uronic acid; about 0.003% to about 0.3% hexozamine; about 0.001 to about 0.1% acetyl; about 0.001% to about 0.1% sulfate; about 0.02% to about 5.0% of at least one mucopolysaccharide; about 0.05% to about 0.5% of at least one fibroblast growth factor; about 0.01% to about 0.1% of at least one enzyme; about 0.01% to about 1.0% hyaluronic acid; about 0.001% to about 0.1% of at least one oxygen carrier; about 0.001% to about 0.1% of at least one high molecular weight protein; about 0.0001 to about 0.01% of at least one trace element; about 0.005% to about 0.1% of at least one high molecular weight antioxidant compound; about 0.005% to about 0.1% of at least one low molecular weight antioxidant compound; and/or about 80% to about 98% water. Suitably, the gastropod biological fluid is obtained from Helix Aspersa Müller.

The at least one mucopolysaccharide may include glycosaminoglycans (GAGs) and proteoglycans (PGs) containing carbohydrate moieties including chondroitin sulfate (CS), dermatan sulfate (DS), heparin (HP) and heparan sulfate (HS). Suitably, the mucopolysaccharide is a novel glycosaminoglycan produced from Helix Aspersa Müller having a molecular weight of about 29,000 Da, calculated based on viscometery, and a uniform repeating disaccharide structure of structure of (1→4)-2-acetyl,2-deoxy-1-D-glucopyranose (1→4)-2-sulfo-α-L-idopyranosyluronic acid. This polysaccharide represents a unique and believed novel glycosaminoglycan. It is related to the heparin and heparan sulfate families of glycosaminoglycans, but is distinctly different from all known members of these classes of glycosaminoglycans. The structure of this polysaccharide has an adjacent N-acetylglucosamine and 2-sulfo-iduronic acid residues. The combination of a core protein and a specific glycosaminoglycan generates a unique proteoglycan with a precise developmental patter. This glycosaminoglycan represents approximately about 3% to about 5% of the dry weight of the snail's soft body tissues, suggesting important biological roles for the survival of this organism. The gastropod glycosaminoglycan tightly binds divalent cations, such as copper (II). The mucopolysaccharide is at least about 0.02%, at least about 0.2%, at least about 0.3%, at least about 0.5%, at least about 0.8, at least about 1%, at least about 2%, at least about 3%, or at least about 4% of the composition of the gastropod biological fluid.

The fibroblast growth factors (FGF's) are a family of structurally related polypeptide growth factors, currently consisting of 23 members having a heparin-binding domain. FGFs are known to play a role in angiogenesis, wound healing, and embryonic development. The at least one fibroblast growth factor (FGF) of the present technology may be chosen from a group comprising, for example, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23 or a combination thereof. Suitably, at least one fibroblast growth factor utilized in the practice of the present technology is FGF1, FGF4, FGF7, FGF10 or a combination thereof. The unique gastropod biological fluid of the present technology contains at least about 0.005%, at least about 0.01%, at least about 0.05%, at least about 0.08%, at least about 0.1%, at least about 0.2%, at least about 0.5% by weight of the composition of at least one fibroblast growth factor.

The unique biological gastropod fluid of the present technology also includes one or more enzymes. The enzyme may include, for example, gelatinases, proteoglycanases, collagenases or combinations thereof. Gelatinases include any proteolytic enzyme that allows a living organism to hydrolyse gelatin, including but not limited to, MMP2 and MMP9. Proteoglycanases belong to the family of matrixmetalloproteinases (MMPs) which are zinc dependent endopeptidases. They are able to degrade all kinds of extracellular matrix proteins. Suitable proteoglycanases include, but are not limited to MMP-23a, MMP-2, MMP23B, MMP-7, MMP-26. Collegenases include any enzyme that break the peptide bonds of collagen. Collegenases are thought to assist in destroying extracellular structures in pathogens such as bacteria, and therefore are produced in response to tissue damage or an immune response. The one or more enzymes comprise at least about 0.005%, alternatively at least about 0.01%, alternatively at least about 0.02%, alternatively at least about 0.05%, alternatively at least about 0.08%, or alternatively at least about 0.1% by weight of the composition of the gastropod biological fluid.

The gastropod biological fluid also contains at least one oxygen carrier, e.g. hemocyanins, such as copper haemocyanin. Hemocyanins are high molecular weight oxygen-carrying proteins. The oxygen-carrying proteins are thought to play a role in reversing oxidative damage and promoting cell proliferation and regeneration in skin cells. The gastropod biological fluid contains at least about 0.001%, alternatively at least about 0.01%, alternatively at least about 0.02%, alternatively at least about 0.05%, alternatively at least about 0.08%, or alternatively at least about 0.1% of at least one oxygen carrier.

The gastropod biological fluid further includes at least one higher molecular weight protein. Higher molecular weight proteins found in biological fluid may include mucin or mucus glycoproteins. Mucin or mucus glycoproteins are a family of polydisperse molecules designed to carry out multiple tasks at mucosal surfaces throughout the body. Mucins are high molecular weight epithelial glycoproteins with a high content of clustered oligosaccharides O-glycosidically linked to tandem repeat peptides rich in threonine, serine and proline. They are rich in cysteine residues involved in sub unit crosslinking that form a macromolecular complex. They also contribute to the mucus gel barrier and are part of the dynamic, interactive, mucosal defensive system. Over the years, several studies carried out on mucus glycoprotein from many organs have suggested that these macromolecules consist of subunits held together by interchain disulphide bonds and further stabilized by non-covalent interactions. Interestingly, the gastropod biological fluid of the present technology comprises approximately at least about 0.001%, alternatively at least about 0.005%, alternatively at least about 0.01%, alternatively at least about 0.05%, or alternatively at least about 0.08% by weight of at least one high molecular weight protein.

The gastropod biological fluid further includes at least one trace element. Trace elements include, for example, copper (Cu), zinc (Zn), iron (Fe), calcium (Ca²⁺) or combinations thereof. Trace elements comprise about 0.0001%, alternatively at least about 0.0005%, alternatively at least about 0.001%, alternatively at least about 0.005%, or alternatively at least about 0.01% by weight of the biological fluid.

The gastropod biological fluid further may contain at least one high molecular weight antioxidant compound. Antioxidants are molecules capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents, including but not limited to, thiols or polyphenols. The gastropod biological fluid contains about 0.005%, alternatively at least about 0.008%, alternatively at least about 0.01%, alternatively at least about 0.05%, or alternatively at least about 0.1% by weight of at least one higher molecular weight antioxidants. Further, cosmeceuticals, compositions or formulations comprising the gastropod biological fluid may also further comprise at least one additional antioxidant compound. Any suitable naturally derived antioxidant compounds known in the art may be used, including but not limited to, olive oil derivatives, oleic acid, palmitic acid, uric acid, carotenoids, ubichinones, lipoic acid, vitamin C, vitamin E, phenoic compounds, resveratrol, beta carotene, selenium, high molecular weight antioxidants, low molecular weight antioxidant compounds, derivatives thereof and combinations there of. The cosmeceutical, composition, or formulation may include the antioxidant compound at about 0.01% to about 10.0%, more suitably about 0.1% to about 5%, more suitably about 0.5% to about 3% by weight of the total composition. The antioxidants may include about 0.01%, about 0.05%, about 0.1%, about 0.2%, about 0.5%, about 0.8%, about 1.0%, about 1.2%, about 1.5%, about 1.8%, about 2.0%, about 2.2%, about 2.5%, about 2.8%, about 3.0%, about 5.0%, about 7.0%, about 8.0%, about 10.0% by weight of the cosmeceutical, composition or formulation.

The gastropod biological fluid also contains about 0.005%, at least about 0.008%, at least about 0.01%, at least about 0.05%, at least about 0.1% by weight of at least one low molecular weight antioxidant compound. Low molecular weight antioxidant compounds are an important part of the antioxidative defense mechanisms of cells and organisms. The main low molecular weight antioxidants, include, but are not limited to, uric acid, ubichinones, lipoic acid, vitamins C and E, carotenoids, and phenolic compounds.

The gastropod biological fluid may be stored before usage and final formulation/composition/cosmeceutical processing. The fluid may be rapidly frozen at between about −10° C. to about −30° C., preferably about −15° C. to about −20° C. Large amounts of the biological fluid may be stored for over a year by freeze drying. The biological fluid is thermosensitive and can be denatured at about 60° C. to 80° C., and the regenerative and antibiotic properties are lost if heated above about 90° C. Preferably, the biological fluid is stable in compositions when stored at temperatures between about −20° C. to about 70° C.

In some embodiments, the gastropod biological fluid may be used in an effective amount in a cosmeceutical. A “cosmeceutical” is a composition comprising a combination of a cosmetic and biological and/or pharmaceutical product, such as an anti-wrinkle cream and a sunscreen. Preferably, the cosmeceutical of the present technology provides a beneficial property to the skin. Beneficial properties include, but are not limited to, maintaining healthy skin; moisturizing; healing and preventing scarring; healing of sun damaged skin; preventing premature aging and wrinkles, inducing skin regeneration and wound healing; inducing skin repair; reducing oxidative stress; whitening skin pigmentation or lessening skin color; restoring tissue plasticity; opening clogged pores; treating ingrown hairs, razor bumps, dryness and photoaging; anti-inflammatory effects; reducing acne scarring; fighting acne breakouts; or fighting scalp and skin infections. Suitable cosmeceuticals which may include gastropod biological fluid of the present technology preferably include anti-aging, anti-wrinkle, acne treatments, wound patches, wound salve, whitening compositions, and antimicrobial compositions.

An “effective amount” of the gastropod biological fluid is an amount that provides the desired beneficial outcome. The effective amount of the gastropod biological fluid used in a cosmeceutical of the present technology depends on the delivery system being used and the type of application. An effective amount of the gastropod biological fluid includes at least about 1%, at least about 5%, at least about 10%, at least about 15%, at least about 20%, at least about 25%, at least about 30%, at least about 35% by weight of the cosmeceutical. The gastropod biological fluid may be about 1%, about 2%, about 4%, about 6%, about 8%, about 10%, about 12%, about 14%, about 16%, about 18%, about 20%, about 22%, about 24%, about 25%, about 26%, about 28%, about 30%, about 32%, about 33%, about 34%, about 35% by weight of the cosmoceutical by weight.

The cosmeceutical may include an effective amount of a gastropod biological fluid and a delivery vehicle capable of being applied to the skin. Suitable delivery vehicles include, but are not limited to, a cream, a solution, a gel, an oil, a suspension, a dispersion, an ointment, a membrane, a patch, a powder, a capsule, a soap, a suppository, a derivative thereof or a combination thereof.

The amount of gastropod biological fluid may depend on the delivery vehicle being used. In one embodiment, for example, a cream used for treatment of the face, hands or body may include about 15% to about 20% gastropod biological fluid. In another embodiment of a liquid to soak hand and feet or wash the body in a bath, the composition, cosmeceutical, or formulation may contain about 1% to about 5% gastropod biological fluid. In a further embodiment of an oil for topical treatment of specific skin areas, the composition, cosmeceutical, or formulation may include about 30% to about 35% gastropod biological fluid. In yet another embodiment, the composition, cosmeceutical, or formulation may be a powder to be applied to a would area or specific skin areas containing about 15% to about 20% of the gastropod biological fluid. In another embodiment, the composition, cosmeceutical, or formulation may be a mask to cover a large wound or a face mask that includes about 20% to about 25% gastropod biological fluid. In yet another embodiment, the cosmeceutical may be a membrane to cover part of the face or body area or a wound area that includes about 20% to about 25% gastropod biological fluid. As a patch or strip which may control release of the active ingredients, the cosmeceutical may include about 15% to about 25% gastropod biological fluid. In a still further embodiment, the cosmeceutical may include a pill or a capsule for oral administration of the active ingredients that includes about 25% to about 30% by weight of the total cosmeceutical of the gastropod biological fluid. In yet a further embodiment, the cosmeceutical may be a soap to wash hand, face or body which includes the gastropod biological fluid at about 1% to about 10%, more suitably about 1% to about 3% by weight of the total composition.

Further, the cosmeceutical may further include at least one olive oil or olive oil derivative. Olive oil derivatives include, but are not limited to, oleic acid, palmitic acid, and other fatty acids, squalene, sterols including phytosterol and tocosterols, tyrosol, hydroxytryrosol including oleocanthal and oleuropein, Peg-4 olivate, surfactant sorbitan olivate, derivatives thereof, or combinations thereof. Olive oil derivatives contain potent antioxidant properties along with moisturizing and immune stimulating properties. The compositions include about 0.01% to about 10%, more suitable about 0.5% to about 5% by weight of at least one olive oil derivative. The at least one olive oil derivate may be about 0.08%, about 0.1%, about 0.2%, about 0.3%, about 0.5%, about 0.8%, about 1.0%, about 1.2%, about 1.5%, about 1.8%, about 2.0%, about 2.2%, about 2.3%, about 2.4%, about 2.5%, about 2.6%, about 2.8%, about 3.0%, about 3.2%, about 3.4%, about 3.6%, about 4.0%, about 4.2%, about 4.5%, about 4.8%, about 5.0%, about 7.0%, about 10.0% of the total weight of the cosmeceutical, composition, or formulation of the present invention. The olive oil derivative may suitably be combination of peg 4 olivate, squalane, and sorbitan olivate. In one suitable embodiment, the at least one olive oil derivative includes about 0.2% to about 2.0%, more suitably 0.8% to about 1.0% peg 4 olivate; about 0.2% to about 2.0%, more suitably about 0.8% to about 1.0% squalane; and about 0.2% to about 2.0%, more suitably about 0.8% to about 1.0% sorbitan olivate.

In some embodiments the cosmeceutical further includes squalane at about 0.01% to about 10%, more suitably about 0.1% to about 2%, more suitably about 0.8% to about 1.2% based on the total weight of the cosmeceutical, composition, or formulation. Squalane is a natural organic compound obtained from mainly from shark liver oil or olive oil, but also found in botanic sources such as amaranth seed, rice bran, wheat germ, and olives. Squalene is used in the cosmeceutical as a natural moisturizer and antioxidant which penetrates skin quickly and does not leave a greasy residue.

In some embodiments, the cosmeceutical further includes hyaluronic acid. Hyaluronic acid may comprise about 0.01% to about 10%, more suitably about 0.1% to about 5%, more suitably about 0.5 to about 2%, more suitably about 0.8% to about 1.0% of the total weight of the composition. Hyaluronic acid is a non-sulfated glycosaminoglycan distributed widely throughout connective, epithelial, and neural tissues. It is one of the main components of extracellular matrix and contributes to cell proliferation and migration, participates in a number of cell surface receptor interactions and is involved in tissue repair. Hyaluronic acid has been shown to be an effective scavenger of free radicals and may also be effective when scavenging oxidants caused by ultraviolet radiation. It plays a role in inducing tissue healing, notably post-surgically, e.g. cataract surgery. Within the composition, the hyaluronic acid may play a role in tissue healing and reducing oxidative stress on the skin. In some embodiments, hyaluronic acid and squalene are included with the gastropod biological fluid to form the desired cosmeceutical.

In further embodiments, the cosmeceutical, composition or formulation may include a preservative. The preservative is an ingredient added to the cosmeceutical, composition, or formulation to prevent the growth of microorganisms including bacteria, viruses, yeast and fungi, and to protect the cosmeceutical from damage caused from air and light. The cosmeceutical may include the preservative at about 0.01 to about 10%, more suitable about 0.1 to about 5%, more suitably about 0.5% to about 5%, more suitably about 0.8% to about 1.2% based on the total weight of the composition. Particularly suitably preservative includes phenoxyethanol, ethylhexylglycerin, derivatives thereof, or combinations thereof. Phenoxyethanol is an organic chemical compound, a glycol ether bactericide. Ethylhexylglycerin (1-(2-ethylhexyl)-glycerinether) is an enhancer of the action of preservatives, inhibits bacterial growth and is a mild humectant and skin emollient. Any suitable preservative known in the art to be used in cosmetics may be used. Other alternative preservatives include paraben. Further, the preservative may be a combination of botanical extracts. Suitable botanical extracts include, for example, organum vulgare leaf extract, thymus vulgaris extract, cinnamomum zeylanicum bark extract, rosmarinus officinalis leaf extract, lavandula augustifolia flower extract, citrus medica limonum peel extract, menthe piperita leaf extract, hydrastis Canadensis root extract, olea europaea leaf extract, derivatives thereof, or a combination thereof.

The cosmeceutical, composition or formulation may further comprises at least one solvent. A solvent is a liquid that dissolves a solid, liquid or gas into a resulting solution. Suitable solvents include, but are not limited to water, glycerin, alcohols, derivatives thereof and combinations thereof. Glycerin is a colorless, dense sugar alcohol that can also dissolve easily in alcohol or water. Glycerin also is highly “hygroscopic” which means that it absorbs water from the air. Glycerin provides the compositions with means of improving smoothness, providing lubrication and as a humectant. The amount of glycerin depends on the delivery system of the cosmeceutical, composition or formulation. Suitably, the cosmeceutical, composition or formulation includes the solvent at about 0.1% to about 99%, alternatively about 10% to about 90% of the total weight. The cosmeceutical may include the solvent at least about 0.1%, about 1.0%, about 5%, about 8%, about 10.0%, about 12.0%, about 15.0%, about 18.0%, about 20.0%, about 22.0%, about 24.0%, about 25.0%, about 28.0%, about 30.0%, about 32.0%, about 35.0%, about 36.0%, about 38.0%, about 40.0%, about 42.0%, about 44.0%, about 48.0%, about 50.0%, about 55.0%, about 58.0%, about 60.0%, about 62.0%, about 64.0%, about 66.0%, about 68.0%, about 70.0%, about 72.0%, about 74.0%, about 76.0%, about 78.0%, about 80.0%, about 82.0%, about 84.0%, about 86.0%, about 88.0%, about 90.0%, about 92.0%, about 94.0%, about 96.0%, about 98.0% by weight of the composition, cosmoceutical or formulation.

In some embodiments, the cosmeceutical, composition or formulation may include an abrasive agent. Suitably abrasive agents provide a sloughing outcome of dead cells or tissue and other debris without causing damage to the skin. Suitable abrasive agents are known in the art and include, but are not limited to, corudrum oxide. The cosmeceutical, composition or formulation may include about 0.0005% to about 0.05%, more suitably about 0.001% to about 0.01% by weight of the composition.

In some embodiments, the cosmeceutical, composition or formulation may include an aromatic essence. An aromatic essence may provide an aroma to the formulation, which include, but are not limited to, for example, vanilla, jasmine, violet, rose, lavender, chamomile, peach, strawberry, coconut, green tea, mint, citrus, etc. The cosmeceutical, composition or formulation may include the aromatic essence at about 0.005%, alternatively at about 0.01%, alternatively at about 0.05%, alternatively at about 0.1%, alternatively at about 0.5%, alternatively at about 0.8%, alternatively at about 1.0%, or alternatively at about 2.0%.

In a particular embodiment, the cosmeceutical, composition or formulation may includes about 5.0% to about 10.0% stearic acid, about 0.1% to about 1% single stearic acid glyceride, about 1% to about 10% of at least one oil, about 0.1% to about 1% octadecanol, about 5% to about 15% glycerin, about 10% to about 20% gastropod biological fluid, about 0.1% to about 1.0% potassium hydroxide, about 0.1% to about 2.0% fragrance essence, and about 0.1% to about 2.0% of a preservative comprising a combination of phenoxyethanol and ethylhexygycerin. The one or more oil may include white oil, silicon oil, a derivative thereof, or a combination thereof. Suitably the composition, cosmeceutical, or formulation includes at least one oil containing about 4.0% to about 5.0% white oil and about 1.0% to about 2.0% silicon oil based on total weight of the composition. This cosmeceutical may further comprise water in an amount sufficient to bring the composition to approximately 1000 grams.

In another particular embodiment, the cosmeceutical, composition or formulation may include about 0.1% to about 1.2% peg 4 olivate, about 0.1% to about 1.2% sorbitan olivate, about 0.1% to about 1.2% squalane, about 10.0% to about 20.0% gastropod biological serum, about 0.1% to about 2.0% hyaluronic acid, about 10% to about 20% glycerin, about 0.1% to about 2% a preservative (e.g., phenoxyethanol and ethylhexygycerin) and about 0.1% to about 1% corundum oxide based on the total weight of the composition.

In yet another embodiment, the cosmeceutical, composition, or formulation comprises about 2.4% to about 3.0% of at least one olive oil derivative, about 10% to about 15% gastropod biological fluid, about 0.8% to about 1.2% hyaluronic acid, about 10.0% to about 15.0% glycerin, and about 0.8% to 1.2% a preservative (e.g., phenoxyethanol and ethylhexygycerin) based on total weight of the composition. Suitably these embodiments include water to bring the weight of the formulation to approximately 1000 g.

In yet another particular embodiment, the cosmeceutical, composition or formulation may include about 0.01% to about 5.0%, more suitably about 0.1% to about 2.0% rose hip seed oil based on the total weight of the composition. Rose hip seed oil is rich in precursors of vitamin A and essential fatty acids to nourish and moisturize the skin. The cosmeceutical, composition or formulation may further comprise about 5% to about 20.0%, more suitably about 10% to about 15% gastropod biological fluid filtrate, about 0.1% to about 10.0% olive oil derivative based on the total weight of the composition. Olive oil derivative comprises emulsifiers, surfactant and squalane and derivatives thereof that replenish the lipid content of the skin, which include, but are not limited to, those described herein. The cosmeceutical, composition or formulation may further comprises about 0.5% to about 5.0%, more suitably about 1.5% to about 2.5% seaweed extract based on total weight of the composition. Seaweed extract provides oligoelements that promote cell growth. The cosmeceutical further comprises about 0.01% to about 10.0%, more suitably about 0.1% to about 2.0% hyaluronic acid, about 0.1% to about 10%, more suitably about 1.0% to about 4% chamomile. Chamomile provides anti-inflammatory properties and is useful for sensitive skin. Further, the cosmeceutical, composition or formulation may include about 0.1% to about 10%, more suitably about 2.0% to about 5.0% saccharide isomerate based on total weight of the composition. Saccharide isomerate includes a carbohydrate complex similar to complexes found in the skin that promotes the skin to retain water. Further, the formulation may include licorice extract (Glycyrrhiza inflata extract) which has anti-oxidant, anti-inflammatory, anti-irritant, antimicrobial, and sebum regulator activities. The licorice extract may include about 0.5% to about 10%, more suitably about 1.0% to about 5.0% based on the total weight of the composition. Further the composition may include about 0.1% to about 10%, more suitably about 1% to about 5% based on total weight of the composition of a preservative derived from natural botanical extracts. These botanical extracts provide antimicrobial, antibacterial, antifungal, anti-yeast and/or antiviral properties. Suitably botanical extracts include, but are not limited to Origanum Vulgare (Oregano) Leaf Extract, Thymus Vulgaris (Thyme) Extract, Cinnamomum Zeylanicum (Cinnamon) Bark Extract, Rosmarinus Officinalis (Rosemary) Leaf Extract, Lavandula Augustifolia (Lavender) Flower Extract, Citrus Medica Limonum (Lemon) Peel Extract, Mentha Piperita (Peppermint) Leaf Extract, Hydrastis Canadensis (Golden Seal) Root Extract, and Olea Europaea (Olive) Leaf Extract.

The cosmeceutical, composition or formulation of the present technology may also include an effective amount of a gastropod biological system and ingredients derived from the traditional Chinese medicine to create novel formulations. Suitable ingredients derived from Chinese medicine include, but are not limited to, oils (e.g., rose oil, emulsion aburagi fruit oil, corn embryo oil, wheat embryo oil, fibert oil, sesame oil), sugars and sugar derivatives (e.g. dextrose, AHA, glycolic acid, glycerins), plant and fruit essence (e.g. Ginko essence, tulip essence, aloe, hamamelis essence, thyme essence, salicylic acid, willow bark essence, essence of carbamide and liquorice, citrus essence, blackberry essence); herbs (e.g. Ganoderma Lucidum, Panax Ginseng); fruits, nuts, vegetable and derivatives thereof (e.g. red guava, mung bean, chestnut, raspberry, ellagic acid, seaweed, orange flower water, flaxseed acid, sunflower seed), vitamins (e.g., vitamin C, vitamin E, ascorbic acid, coenzyme q); detergents (e.g., sodium lauryl or laureth sulfate, TEA-laurylsulfate); emulsifiers (e.g., Cocamide DEA); fungi and fungi derivatives (e.g., Kojic acid); and others (e.g. Sodium C14-16 olefin sulfate C14-16, triethanolamine, honey essence, levorotation-C, amides, jobs of tears, arginine, lactic acid, urea, etc.). These formulations may be used for a variety of skin care treatment products, including but not limited to, wound patches, postoperative healing membranes, rejuvenation essence, antiphlogistics (purification conditioning cream), anti-acne cream, anti-wrinkle cream, moisten facial cleansing soap, shower gel, hand cream, whiten cream, post-partum dressing, hemorrhoids thrombolysis, anti-ultraviolet skin care cream, anti-drying skin-care cream, anti-burn healing cream).

A particular embodiment of a whitening cream includes the gastropod biological fluid, vitamin C, yellow gentian grass, kojic acid, at least one antioxidant (e.g., green tea), and at least one plant extract. Suitable plant extracts include sorbic, occident pear, olive fruit, gingko, ursolic, foreign beech, seaweed, birch, mulberry, Panax, etc.

A particular embodiment of a sunscreen cosmeceutical cream includes the gastropod biological fluid, and at least one UV-blocking agent selected from the group comprising PABA (e.g., Para-aminobenzonic acid, Padimate O, Padimate A, Glyceryl PAA), salicylates (e.g., homosalate, octyl salicylate, trolamine salicylate), cinnamates (e.g., octyl methoxycinnamate, cinoxate); benzophenones (e.g., oxybenzone, dioxybenzone, sulisobenzone), anthranilate, methylanthranilate, physical agents (e.g. xinc oxide 4, red petroleum) and a combination thereof.

A particular embodiment of a therapeutic anti-wrinkle (anti-crease) cream cosmeceutical of the present technology comprises the gastropod biological fluid in combination with, for example, ginko biloba, green tea factors, aloe, biopeptide-C1, vitamin E, placenta prime, ARL against aging factor, and almond

A particular embodiment of a therapeutic acne removing cosmeceutical cream of the present technology includes, for example, the gastropod biological fluid, gentian garlic extract, hop oil, chamomile extract, sunflower extract, tea-tree oil, and calendula.

Any of the above embodiments may further include a wetting agent which makes your skin feel slippery. These wetting agents may include PEG (polyethylene glycol), polysorbates, propylene glycol, polypropylene glycol, butylene glycol, hexylene glycol, isopropyl myristate, derivatives thereof or combinations. Any of the above embodiments may also include additional preservatives to prevent bacteria, mold fungal, parasitic, yeast, or other microorganism from growing. Suitable preservatives may include, for example, quaternium 15, imidazolidinyl urea, ethyparaben, butylparaben, propylparaben, methylparaben.

A particular embodiment of a moisture retention cream cosmeceutical of the present technology includes the gastropod biological fluid, glycerol, HA (hyaluronic acid), PCA-NA (L-Pyrrolidone Carboxylic Acid-Na or Sodium L-pyroglutamate), NMF (Natural Moisturizing Factor), nerve thiamine and collagen protein, carbamide, lactic acid, chitosan, aloe, and seaweed extract. Other suitable additional components include, for example, propylene glycol, polypropylene glycol, butylene glycol, hexylene glycol, caprylic, capric, lauric triglycerides, cyclomethicone, dimethicone, fatty acids, hyaluronic acid, sodium PCA, collagen, ceramide, elastin, lecithin, derivatives thereof or combinations thereof.

The cosmeceuitcal may also be prepared as a therapeutic cream as described in the following examples. Such a therapeutic cream may be use to (i) treat types of burns including thermal and chemical burns, (ii) prevent or treat radiodermatitis, and (iii) to prevent or treat sunburn.

In another embodiment, the cosmeceutical of the present technology may be a nourishing face or hand cream for use on normal, dry or oily skin. It is believed that the gastropod biological fluid repairs the epithelium and rejuvenates the skin. It is also believed that the cream also serves as an exfoliate, eliminating the outer dead skin layer (epithelial corneal layer) and encouraging the growth of new skin. The use of panthotenic acid and heparin in the cream is optional, but preferred because they increase the restorative and penetrating actions. The cream tones and revitalizes the skin and may be used for treatment of rosacea, eczema, dermatitis and very dry skin conditions.

In a still further embodiment, a cosmeceutical of the present technology may be a clarifying and moisturizing liquid for dry, normal or oily skin. The resulting liquid cleanses and smoothes the skin and heightens the transparency of the complexion. It serves also as a base for make-up (e.g., powder) and protects the skin from the harmful action of cosmetic powders which consist of minerals which have a hardness value of 1 on the Mohr scale. A suitable formulation comprises about 2 ml of gastropod biological fluid in about 200 g total product. Suitably, the clarifying and moisturizing liquid preferably includes about 0.5%, alternatively about 1.0%, alternatively about 2%, alternatively about 5%, alternatively about 10%, alternatively about 15% of the gastropod biological fluid of the present invention.

In another embodiment, a cosmeceutical of the present technology may be a toning and cleansing liquid. The liquid is recommended for use on delicate skin or skin where acne is present. It produces an intense refreshing feeling and a soothing, restful sensation

In yet another embodiment, a cosmeceutical of the present technology may be an anti-wrinkle cream for daily or weekly use. The cream is easy to apply and best if used only on areas where wrinkles exist. The gastropod biological fluid active ingredient is believed to be effective in reducing wrinkling, whereas the other components produce an astringent action. The effect of the cream lasts at least one week after each application.

Further, a cosmeceutical of the present technology may be used as a sunscreen or as a treatment for sunburn. Suitably, the sunscreen must be applied before exposure to the sun. For treatment of sunburn, the cream may be applied every six hours until the symptoms are gone. Further, the cream may be used as a pigment corrector for sun & age spots and melasma, which include the gastropod biological fluid along with a Rumex botanical extract, and a biomimetic peptide that takes away hyperpigmentation without any side effects.

Other cosmeceuticals envisaged according to the present technology include a home microdermabrasion cream infused with the skin regeneration activators and micro-crystals to polish away roughness, dull skin, damaged tissues and scars and allow for the enzymes in the serum to dissolve damaged proteins and penetrate deeply within the skin; an oil free gel to prevent and treat acne, rosacea and scars; and a cream for rough, old body scars, pregnancy stretch marks by dissolving deep scar tissues and by releasing their amino-acids for the rebuilding of all the structural elements in charge of the skin's mechanical properties, including firmness, strength, suppleness, and elasticity.

An excipient specially designed for the preservation of biological substances and for use in dermatological and cosmetic treatments is used in Younger™ Biocream commercially available from Pharmacom located in Iowa City, Iowa. One of the advantages of this excipient is that it will accept almost all other active ingredients and it gives the compositions optimum stability. This preferred excipient is made from a polyethylene glycol mixture having a molecular weight range of about 900 to about 1300, cetyl alcohol, glyceryl monostearate, selected mineral oils, calcium carbonate in micropowder form, zinc oxide in micropowder form, and an aqueous phase. The excipient consists essentially of about 10% to about 17%, preferably about 12.0% to about 15.5% of cetyl alcohol and/or its by-products such as the laurate, myristate, and adipate; about 20% to about 50%, preferably about 30 to about 50% of the polyethylene glycol mixture (e.g., a mixture of polyethylene glycol 400, 1500, and 4000); about 3.5% to about 5.8%, preferably about 4% to about 6% of glyceryl monostearate; about 7 to about 12%, preferably about 8 to about 10%, of a mineral oil (preferably paraffin oil); about 5% to about 8%, preferably about 5.5% to about 7%, calcium carbonate in the form of a micropowder; about 0.3-0.6%, preferably 0.32-0.40%, zinc oxide in the form of a micropowder; and an effective amount of an aqueous phase, typically up to about 35%, preferably about 16% to about 30%. The amount of the aqueous base should be an amount which is sufficient to give the composition of the desired consistency. If the amount of the aqueous base is above 35%, a preservative should be used. The aqueous base comprises water, a saline solution [e.g., a solution of 0.9% sodium chloride which contains no pyrogenous matter], or physiological serum. The percentages are by weight and total 100%. The percentages can be easily adjusted with minimal experimentation to provide the desired creamy consistency. The excipient is odorless and white and unaffected by ultraviolet radiation, x-rays, and gamma rays. It can be used in the preparation of creams, milks, pomades and the like. It is believed to have no detrimental effect, even when used over a long period or produce adverse reactions such as allergies or carcinogenic effects. It is also believed the excipient promotes the absorption of the active ingredient through the skin, does not break down between about −20° C. and about +70° C., and is unaffected by humidity, and thus may be used in many climatic extremes. Further, the excipient is stable and not affected by decimeter waves, ultraviolet radiation, gamma radiation of 1.3 MeV, and photons. Preferably, is also unaffected by the container in which it is stored. The importance of this excipient is not based on exotic components, but on their proportions which is the result of the numerous experimental formulations.

Optional ingredients which can be used in the cosmeceuticals for the cases where the aqueous base is greater than 35% include a preservative such as a methyl paraben, an oxidation inhibitor such as sodium bisulfite, and other ingredients typically included in therapeutic compositions.

Optional ingredients which can be used in the compositions, cosmeceuticals, or formulations of the present technology include mineral oils such as paraffin, turtle, or other oils in amounts of about 1% to about 10%, preferably about 2%; boric acid in amounts of about 0.1% to about 0.6%; preferably about 0.3%; chlorophyll in amounts of about 0.25% to about 1.5%, preferably about 1.0%; formol (e.g., about 30% concentration) in amounts of about 0.3% to about 3%, preferably about 0.4%; glycerin in amounts of about 0.1% to about 4.0%, preferably about 0.5%; heparin in amounts of about 0.01% to about 0.4%, preferably about 0.1%; lanolin in amounts of about 0.1% to about 6%, preferably about 0.5%; menthol in amounts of about 0.25 to about 2.5%, preferably about 1%; panthotenic acid in amounts of about 0.01% to about 0.5%, preferably about 0.1%; potassium aluminum sulfate in amounts of about 0.1% to about 1.5%, preferably about 0.4%; copper sulfate in amounts of about 0.01% to about 0.3%, preferably about 0.03%; Vitamin C in amounts of about 0.01% to about 0.5%, preferably about 0.2%; or metallic iodine in amounts of about 0.1% to about 1.1%, preferably about 0.6%. The above percentages are by weight with the weight of the total cosmetic composition being 100% by weight.

The presently described technology and its advantages will be better understood by reference to the following examples. These examples are provided to describe specific embodiments of the present technology. By providing these examples, the inventors do not limit the scope and spirit of the present technology. It will be understood by those skilled in the art that the full scope of the presently described technology encompasses the subject matter defined by the claims appending this specification, and any alterations, modifications, alternatives, or equivalents of those claims.

### EXAMPLES

### Example 1

### Collecting and Refining Gastropod Biological Fluid

Helix Aspersa Müller originated from France and were grown on protein-rich food. Helix Aspersa Müller snails were centrifuged at 2 G for 10 minutes with 3 pulsations. A pulsation was performed by accelerating the centrifuge to 2G, decelerating the centrifuge and re-accelerating the centrifuge to 2 G. During each pulsation, the centrifuge was accelerated to 2 G, decelerated and re-accelerated to 2 G. After centrifugation, the fluid secretion of the snail was collected by use of a straw for removing the fluid from the snail and collected into a centrifuge-compatible tube. The fluid secretion was centrifuged at 2000 rpm for 10 minutes to remove any large particles. The supernatant was decanted into a cylinder containing a 0.1 μm Millipore filter. The cylinder was hermetically sealed with a closure bearing a connection to a compressed air system to facilitate filtration. The filtrate was then used as the gastropod biological fluid of the presently described technology.

### Example 2

### Breakdown of Components of Gastropod Biological Fluid

To assess the chemical make-up of the gastropod biological fluid, Helix Aspersa Müller snails were processed to determine their GAG (glycosaminoglycan) concentration. The shell of the snail was removed, and the whole soft body was defatted using three, 24-hour extractions with acetone. The fat-free dried snail was cut into a fine powder using a razor blade. Approximately 4 g of dried, defatted, pulverized powder was suspended into 40 ml of 0.05 M sodium carbonated buffer (pH 9.2). The suspension was shaken for 48 hours at 200 rpm at 60° C. after adding 2 ml of Alcalase (2.4 Anson units/g). The digestion mixture was cooled to 4° C., and trichloroacetic acid was added to a final concentration of 5%. The sample was mixed, allowed to stand for 10 min, and then centrifuged for 20 min at 8000×g. The supernatant was recovered by decanting. Three volumes of 5% potassium acetate in ethanol were added to one volume of supernatant. After mixing, the suspension was stored overnight at 4° C. and then centrifuged for 30 min at 8,000×g. The supernatant was discarded, and the precipitate was washed with absolute alcohol. The precipitate (1 g) was dissolved in 40 ml of 0.2 M NaCl and centrifuged for 30 min at 8,000×g, and insoluble material was discarded. To the resulting material, 0.5 ml of cetylpyridinium chloride (5%) was added, and the resultant precipitate was collected by centrifugation. That precipitate was then dissolved in 10 ml of 2.5 M NaCl, 5 volumes of ethanol was added, and the precipitate was centrifuged for 30 min at 10,000×g. The precipitate was further dissolved in water and dialyzed against 100 volumes of water, and the dialyzed fraction was freeze-dried to obtain 0.18 g of GAG gastropod fluid as a white powder filtrate.

### Analysis of the Physical Properties of the Intact Polysaccharide

Polysaccharide GAG from gastropod biological fluid (200 μg) was thoroughly dried under P₂O₅ in vacuo and dissolved in 0.5 ml of methanolic 1 M HCl using a screw-capped tube with a Teflon-lined cap. Nitrogen gas was bubbled through the solution for 15 seconds, and then the tube was sealed. After methanolysis for 24 hours at 80° C., the acid solution was neutralized by the addition of 0.15 ml of pyridine. Re—N-acetylation was carried out by the addition of 0.1 ml of acetic anhydride. This mixture was kept at room temperature for approximately 1 hour. The sample solution was then evaporated with nitrogen gas flow at 35° C. The residue was dried for 16 hours in vacuo over P₂O₅. Finally, the sample was trimethylsilylated with 50 μl of silylating reagent (pyridine/N,O-Bis-(trimethylsilyl) trifluoroacetamide, 1:2 (v/v)) for 30 min at room temperature. Compositional analysis by gas chromatography (GC) was performed using a capillary column AT-1, 0.53 mm×30-m (1.5-μm thickness), from Alltech Associates located in Deerfield Ill. on a Shimadzu (Kyoto, Japan) gas chromatograph, model GC-14A, with a flame ionization detector, equipped with Shimadzu Chromatopac CR501 integrating recorder. The injection port temperature and the detector temperature were 270° C. and 280° C., respectively. For the analysis of mixtures of monosaccharides, the oven temperature was programmed 120-260° C. at 10° C./min.

Analysis of Oligosaccharides Formed from Lyase Treatment of GAGs

The composition of disaccharides and oligosaccharides produced from intact polysaccharide by each heparin lyase was analyzed by capillary electrophoresis (CE) using a Dionex Capillary Electrophoresis system (commercially available from Dionex Corporation in Sunnyvale) with advanced computer interface, model I, equipped with high voltage power supply capable of constant or gradient voltage control using a fused silica capillary from Dionex Corporation (Sunnyvale, Calif.). The CE system was operated in the reverse polarity mode by applying the sample at the cathode and run with 20 mM phosphoric acid adjusted to pH 3.5 with 1 M dibasic sodium phosphate as described previously. The capillary (75-μm inner diameter, 375-μm outer diameter, 68 cm long) was manually washed before use with 0.5 ml of 0.5 M sodium hydroxide followed by 0.5 ml of distilled water, and then 0.5 ml running buffer. Samples were applied using gravity injection (20 seconds) by hydrostatic pressure (45 mm), resulting in a sample volume of 9.2 ml. Each experiment was conducted at a constant 18,000 V. Data collection was at 232 nm. Peaks were identified by co-injection with disaccharide and oligosaccharide standards prepared and characterized in our laboratory. The size of products formed on treating gastropod GAG with heparin lyase II was determined by gel permeation chromatography on a Sephadex G-50 (superfine) column (1.5×25 cm) eluted with 0.2 M sodium chloride and monitored using a carbazole assay. The disaccharide product of heparin lyase II treatment was prepared for analysis by semipreparative strong anion exchange HPLC and desalted by chromatography on a 5-cm×0.5-m Bio-Gel P-2 column. These results showed a complex and unique make-up of the polysaccharides in gastropod biological fluid of the present technology.

### Gradient PAGE Analysis

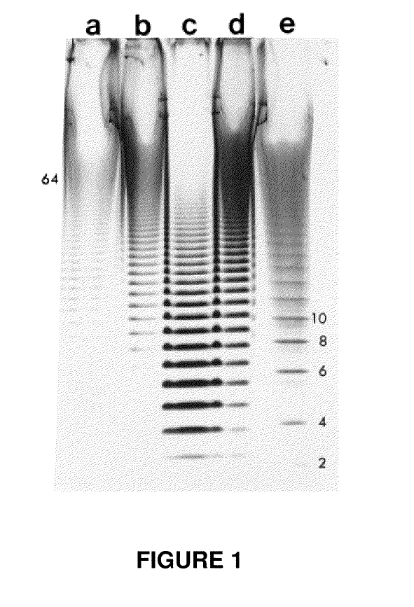

Digestion of the gastropod GAG was digested with heparin lyase I, heparin lyase II, or heparin lyase III. Gradient PAGE was performed on a polyacrylamide linear gradient resolving gel (14×16 cm, 12-22% total acrylamide). Samples were run as untreated snail GAG (lane a), heparin lyase I treated snail GAG (lane b), heparin lyase II treated snail GAG (lane c), heparin lyase III treated snail GAG (lane d) and ladder of heparin oligosaccharide standards (lane e) prepared from bovine lung heparin. Oligosaccharides were visualized by Alcian blue staining. Results can be seen in FIG. 1 showing that gastropod biological fluid of the present technology is a complex make-up of proteoglycans.

Histochemical Staining of the Helix Aspersa Müller to Determine Glycoprotein Concentrations

To determine the protein compositions of Helix Aspersa Müller, thin sections of the Helix Aspersa Müller were fixed in Carnoy's fixative (60% ethanol, 30% chloroform, 10% acetic acid) and 4% paraformaldehyde and embedded in paraplast. Sections were stained by Mallory method modified by Ignesti (Carazzi hemalum, 1% acidic fuchsin, 1% phosphomolybdic acid, Mallory's solution). The following histochemical methods demonstrating proteins, glycoproteins and acidic proteoglycans were used. For proteins the bromophenol blue method, ninhydrin-Schiff, chloramines T-Schiff, performic acid-Schiff, Morel and Sisley, Millon, and Adams reactions, diazoreaction, and Sakaguchi reaction were used. For glycoproteins, the alcian blue-Periodic acid Schiff (AB-PAS) [reaction according to McManus and dimedone-PAS reaction were used. To test for acidic proteoglycans: AB (alcian blue) (pH 1) and AB (pH 2.5) were used. To distinguish between glycoproteins and acidic proteoglycans AB-PAS (pH 1 and 2.5) was used. To distinguish between acidic proteoglycans with different acidities AB-C.E.C. (critical electrolyte concentration) staining, 0.05% AB, inhibition of alcianophilia at graded molarities of 0.5-1 M MgCl₂ in acetic buffer (pH 5.8) weak methylation-AB (pH 2.5); strong methylation-AB (pH 2.5); weak methylation-saponification-AB (pH 2.5); and strong methylation-saponification-AB (pH 2.5) were used. Acid hydrolysis in 0.1 M acetate buffer (pH 2.5) for 4 h at 60° C. followed by AB (alcian blue) (pH 2.5) also without saponification. Methods of enzymatic digestion: PAS-amylase; AB-neuraminidase (pH 2.5, from Clostridium perfrigens) with and without saponification; testicular hyaluronidase. To measure specific sugar residues, biotinylated lectins and avidin-biotinperoxidase method (ABC) was employed. The biotinylated lectins used, their origin, specificity and inhibiting sugars are investigated. To reveal nNOS, tissue sections were incubated overnight at 4° C. in a humid chamber with polyclonal rabbit anti-human brain nNOS antibodies, diluted 1:200 (nNOS type I antibodies; code 606-259-1550). The sections were then washed in PBS (phosphate buffer saline) and incubated for 2 h with a goat anti-rabbit IgG-peroxidase conjugate (1:100). Peroxidase activity was visualized by incubation of the sections for 5-12 min at room temperature in a solution of 0.015% 3,3′-diaminobenzidine in 0.01 M Tris buffer (pH 7.6) containing 0.005% H₂O₂. Negative controls included omission of the primary antibody or its substitution with non-immune rabbit serum.

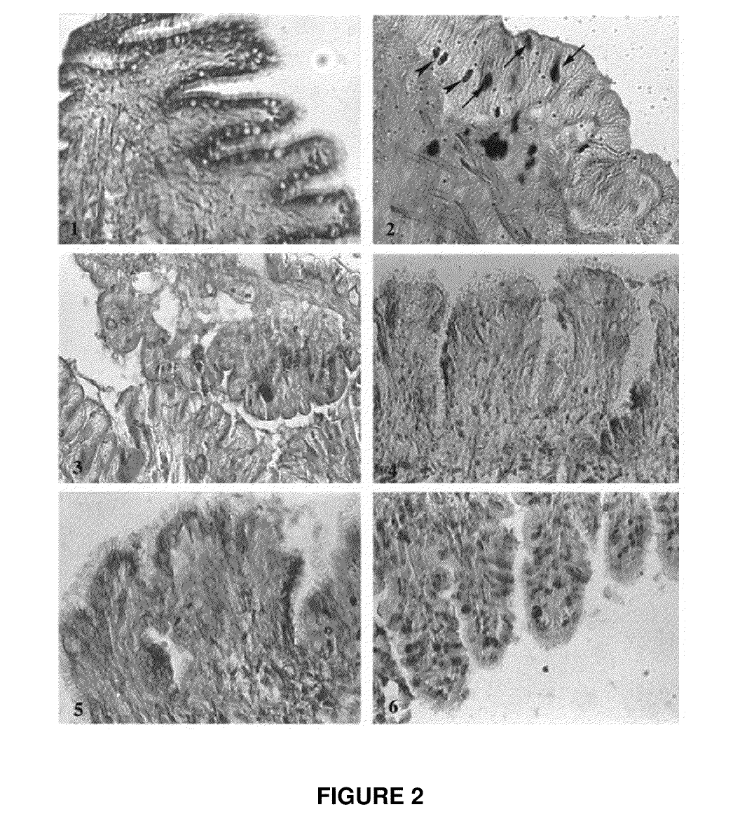

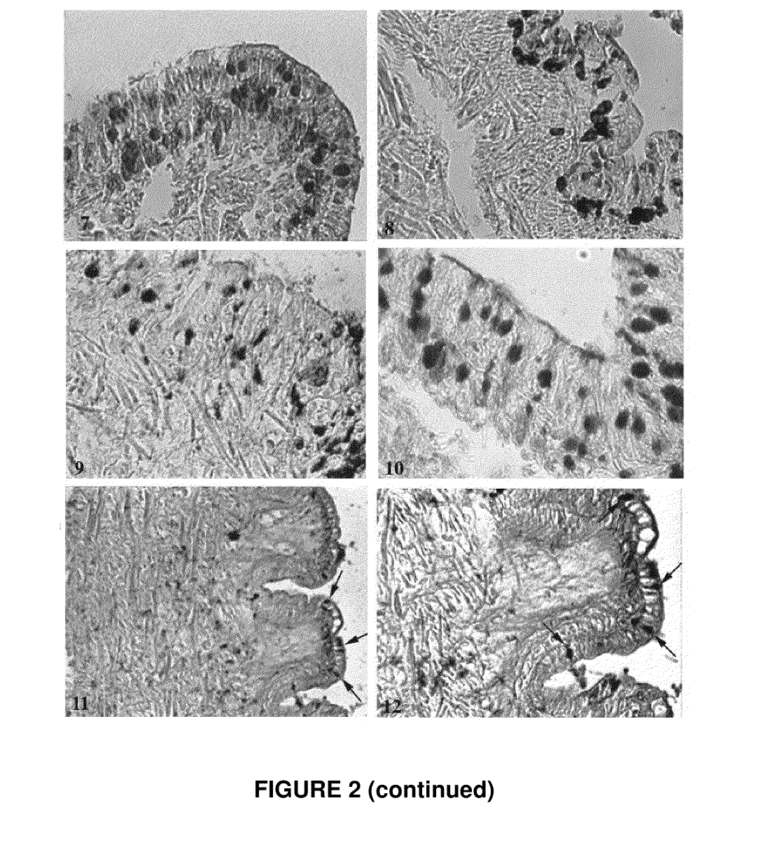

In the mantle of the Helix Apsera Müller snail, the skin consists of rather loose connective tissue, crossed by muscle fibers. On the edge of the mantle, there are four deep folds where the external simple epithelium is composed only of nonciliated cuboidal cells, while in the thickness of the folds there are also goblet cells. It is believed that the mucous cells contain glycoproteins and proteoglycans with acidic groups, see e.g., FIG. 2-2.

The histochemical methods revealed “protein” cells among epithelial cells in both the mantle and foot. The diazoreaction carried out in alkaline medium with Fast blue showed “phenol” cells only in the mantle. PAS positive cells showing the presence of glycoproteins were only found in the mantle. The staining was not present in sections treated with β-amylase, indicating the presence of “glycogen” epithelial cells coexisting with mucous cells in the underlying connective tissue.

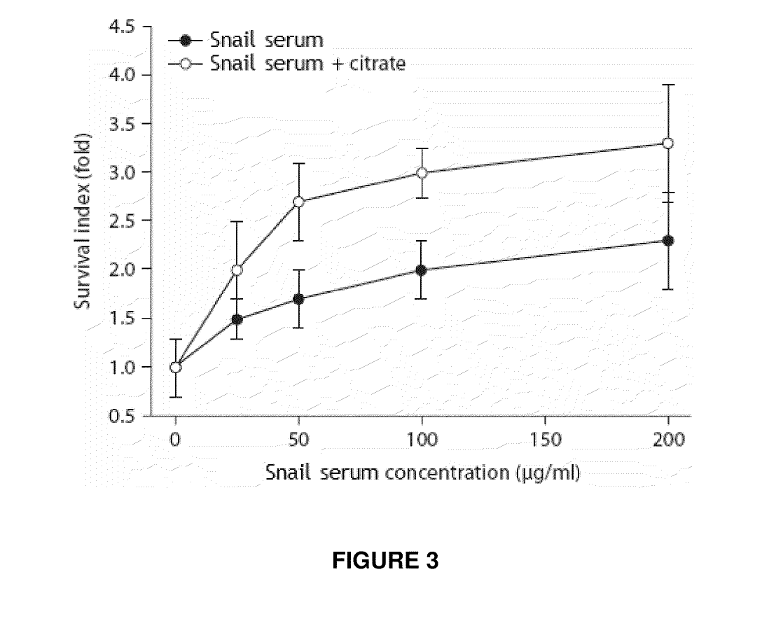

Moreover, the reactions to show acid glycosaminoglycans revealed five types of mucous cells in the mantle: cells with glycoproteins only, with sulphated acidic proteoglycans, with proteoglycans containing carboxyl groups, and with glycoproteins and proteoglycans containing carboxyl groups (see e.g., FIG. 2-3). Some mucous cells of the foot epithelium contained carboxyl acidic proteoglycans (see e.g., FIG. 2-4) others also glycoproteins (see e.g., FIG. 2-5). Sialic acid and hyaluronic acid were not found in the tegument of the mantle and foot. Use of the ConA, PHA-L, PNA, WPA and SBA lectins which all bind different sugar moieties showed that the secretion of the intraepithelial and intradermal mucous cells of the mantle and foot were positive for δ-D-glucose and δ-D-mannose (see e.g., FIG. 2-6), oligosaccharides (see e.g., FIG. 2-7), δ-D-gal and (1-3)-D-Gal-Nac (see e.g., FIG. 2-8), δ-L-fucose (see e.g., FIG. 2-9) and N-acetyl-D-galactosamine or D-galactose (see e.g., FIG. 2-10). In the surface epithelium of the mantle, few neuroendocrine cells were positive for WGA (see e.g., FIG. 2-1). In the mantle epithelium, nNOS-positive nonmucous cells were found (see e.g., FIG. 2-2).

### Example 3

### Gastropod Biological Fluid Induces Fibroblast Proliferation In Vitro and Regulates Fibroblast Cytoskeleton Reorganization

To investigate if the regenerative properties of gastropod biological fluid (labeled SJ, “snail juice”) are related to enhanced cell proliferation, the effect of the biological fluid was assayed on fibroblast proliferation in vitro.

To assay fibroblast proliferation, twenty-five thousand human dermal fibroblasts per well were plated into 12-well plates and treated for a week with the indicated 50 μl or 100 μl of Helix Müller biological fluid (SJ) together with 0.1 mM citrate pH 5.0, 100 U/ml LMW heparin or 1 μM inositol hexasulfate. Monolayers were washed with PBS, fixed in 10% formalin, and rinsed with distilled water. Cells were then stained with 0.1% crystal violet (Sigma) for 30 min, rinsed extensively, and dried. Cell-associated dye was extracted with 2.0 ml 10% acetic acid. Aliquots were diluted 1:4 with H₂O, transferred to 96-well microtiter plates, and the optical density at 590 nm was determined. Values were normalized to the optical density at day 0 for untreated cells. Within an experiment, each point was determined in triplicate; each growth curve was performed at least twice. The growth curves with snail serum with and without citrate are shown in FIG. 3. Interestingly, the biological fluid from the Helix Aspersa Müller increased cell proliferation, which was further enhanced by addition of citrate, which has been shown to induce fibroblast proliferation. This result was further reinforced in that the biological fluid of the present technology increased cell survival upon irradiation with UVA light, which suggests that the observed beneficial effect of biological fluid is due to combined antioxidant and proliferative activities.

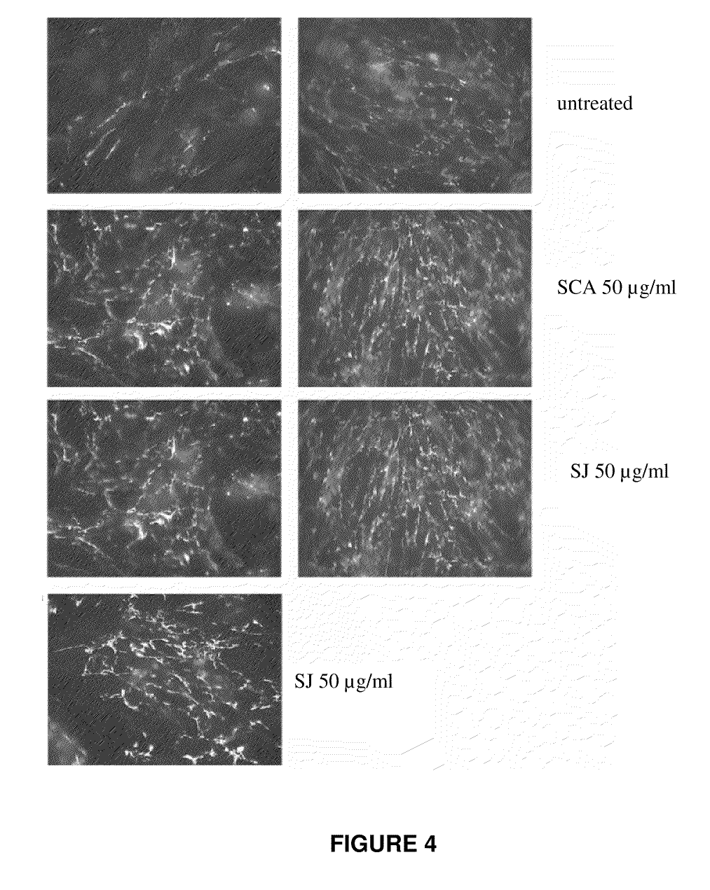

Since extracellular matrix (ECM) assembly is a key event in tissue regeneration, the effects of gastropod biological fluid from Helix Aspersa Müller were assayed. CHO-K1 cells were cultured on glass coverslips using Ham's F-12 medium supplements with penicillin/streptomycin and 10% fetal bovine serum for 24 hours. Cells were then treated in the presence or absence of 5 μg/ml human fibronectin with 50 or 100 μg/ml Helix Aspersa Müller biological fluid (SJ), and fibronectin assembly was monitored after 24 hours by indirect immunofluorescence. Cells were then fixed in chilled methanol for 10 min. Fibronectin was stained with the anti-80 kDa polyclonal antibody followed by incubation with Alexa488-conjugated anti-rabbit antibody. Samples were mounted in Mowiol, and were examined in a Leica DMR photomicroscope with 63× and 100× immersion objectives. Images were processed using Leica QFish software and results are shown in FIG. 4. Gastropod biological fluid from Helix Aspersa Müller induced fibronectin assembly compared to control cells. Interestingly, biological fluid appeared to increase fibronectin secretion since it increased fibronectin deposition by the cells in the absence of exogenous fibronectin. To demonstrate that the increase of fibronectin was not due to fibronectin present in the secretion, biological fluid was analyzed by ELISA and Western blot using a panel of antibodies anti-fibronectin. Biological fluid was found to contain no fibronectin reactivity, as shown by no reactivity with any of the antibodies by Western blot or ELISA. These experiments demonstrated that the observed effect of gastropod biological fluid in fibronectin assembly and secretion is due to its effect on the cells, not on direct deposition of ECM included in the extract. Such an outcome illustrates the unique compositional make-up and properties of the gastropod biological fluid of the present technology.

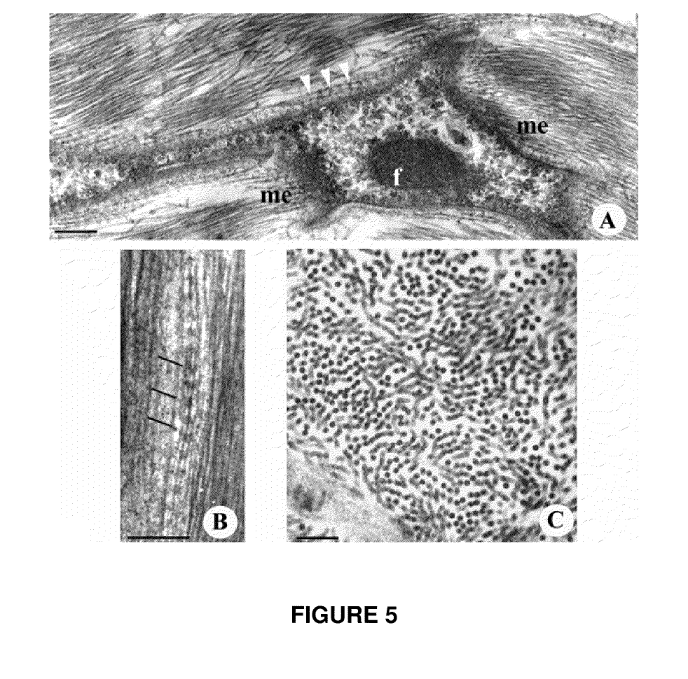

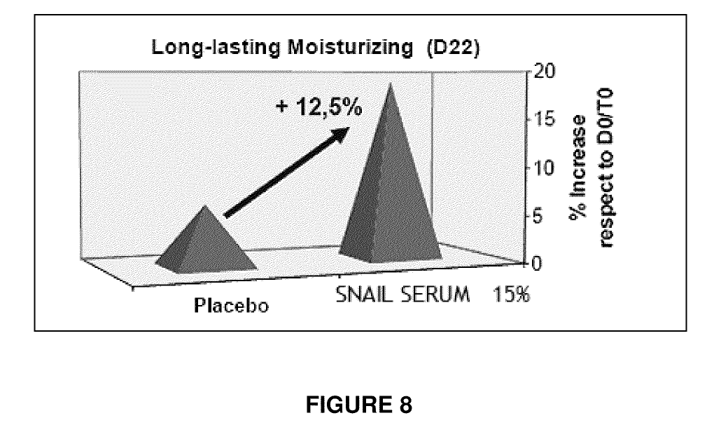

To assay for changes in fibroblast morphology after treatment with Helix Aspersa Müller gastropod biological fluid, human dermal fibroblasts from healthy volunteer donors were cultured for 24 h in the presence or absence of 100 μg/ml of biological fluid. The cells were fixed with 4% formaldehyde in PBS and permeabilized using 0.1% Triton X-100. For F-actin staining, coverslips were incubated with 1 μg/ml Alexa488-conjugated phalloidin (Invitrogen) in PBS for 1 hour at room temperature. Samples were mounted and examined as described previously. At least 200 cells/condition from three independent experiments were examined. Results are shown in FIG. 8. Transverse proteoglycans are visualized by using Alcian Blue theniques shown by arrowhead (A) and bars in (B) in FIG. 5. Secreting fibroblasts (f) and microenv (me) are also labeled (scale bar 0.2 mm). The biological fluid affected the morphology of human dermal fibroblasts, inducing actin reorganization, bundling and microfilament alignment, and resulted in cell elongation. Such an outcome illustrates some of the unique properties of the gastropod biological fluid of the present technology.

### Example 5

### Antioxidative Effects of Gastropod Biological Fluid

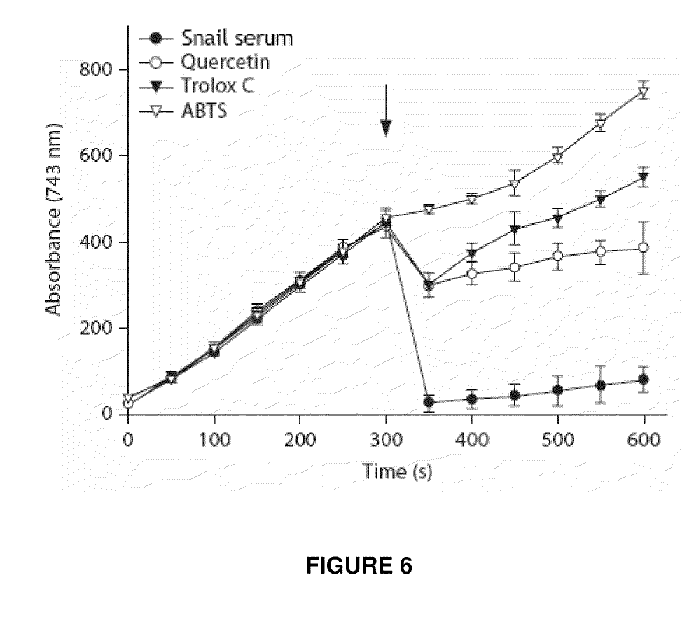

To test the ability of gastropod biological fluid obtained from Helix Aspersa Müller to provide antioxidant properties, a Glutathione-S-Transferase (GST) Colorimetric Assay Kit commercially available from Sigma Chemical Co, St. Louis Mo. was used to assay increase in Glutathione S-transferases (GSTs) activity, a molecule that plays a key role in cellular detoxification and a Superoxide dismutase (SOD) kit commercially available from Calbiochem Co of San Diego Calif. were used. GST and SOD (an antioxidative enzyme) activities were determined according to the kits manufacturers' protocols in 1 mg/ml SCA suspensions. Positive and negative controls were provided in the kits and employed as indicated. An ABTS (2,2′-azino-bis(3-ethylbenzthiazoline-6-sulphonic acid) antioxidant kit commercially available from Vector Labs in Burlingame Calif. was used to test gastropod biological fluid (“SJ”) (100 μg/ml), quercetin (2.5 μM) and Trolox C (10 μM) or vehicle alone at 300 s. Absorbance at 743 nm was measured at different time points before and after addition of the reagent and experiment was preformed in triplicate. FIG. 6 shows representative results of this experiment. This data demonstrates that gastropod biological fluid of the present technology has both antioxidant and free radical scavenging capabilities.

### Example 6

### Preparation of Embodiments of a Cream Cosmeceutical Formulation

The following formulas were made by adding the listed ingredients to make a total of 1000 g of product. The product was thoroughly mixed in a pharmaceutical mixer.

Formula I:
Peg 4 Olivate (about 8 g to about 10 g)
Sorbitan Olivate (from Olive Oil) (about 8 g to about 10 g)
Squalane (from Olive Oil) (about 8 g to about 10 g)
Gastropod biological fluid filtrate (about 100 ml to about 150 ml)
Hyaluronic Acid (about 8 g to about 12 g)
Glycerin (about 110 g to about 130 g)
Phenoxyethanol & Ethylhexygycerin (about 9 g to about 10 g)
Corundum oxide (about 4 g to about 6 g)
Distilled water (about 500 ml) plus enough water to bring the weight of the product to about 1000 g.

Formula 2:
Peg 4 Olivate (about 10 g)
Sorbitan Olivate (from Olive Oil) (about 10 g)
Squalane (from Olive Oil) (about 10 g)
Gastropod biological fluid (about 150 ml)
Hyaluronic Acid (about 10 g)
Glycerin (about 120 g)
Phenoxyethanol & Ethylhexygycerin (about 10 g)
Corundum oxide (about 5 g) and
Distilled water (about 500 ml) plus enough water to bring the weight of the product to about 1000 g.

Formula 3:
Peg 4 Olivate (about 10 g)
Sorbitan Olivate (from Olive Oil) (about 10 g)
Squalane (from Olive Oil) (about 10 g)
Gastropod biological fluid (about 100 ml)
Hyaluronic Acid (about 10 g)
Glycerin (about 120 g)
Phenoxyethanol & Ethylhexygycerin (about 10 g)
Corundum oxide (about 5 g) and
Distilled water (about 500 ml) plus enough water to bring the weight of the product to about 1000 g.

Formula 4:
Stearic acid (about 65 to about 70 g)
Single stearic acid glyceride (about 4 to about 5 g)
White oil (about 45 to about 50 ml)
Silicon oil (about 12 ml to about 15 ml)
Octadecanol (about 4 g to about 5 g)
Glycerin (about 55 ml to about 60 ml)
Gastropod biological fluid (about 100 ml to 150 ml)
Potassium hydroxide (about 2.2 g to about 2.5 g)
Aromatic Essence (about 4.5 ml to about 5 ml
Phenoxyethanol & Ethylhexygycerin (about 9 g to about 11 g)
Distilled water (about 600 ml) plus enough water to bring the weight of the product to about 1000 g.

Formula 5:
Stearic acid (about 70 g)
Single stearic acid glyceride (about 5 g)
White oil (about 50 ml)
Silicon oil (about 15 ml)
Octadecanol (about 5 g)
Glycerin (about 60 ml)
Gastropod biological fluid (about 100 ml)
Potassium hydroxide (about 2.5 g)
Aromatic Essence (5 ml)
Phenoxyethanol & Ethylhexygycerin (about 10 g)
Distilled water (about 625 ml) plus enough water to bring the weight of the product to about 1000 g.

Formula 6:
Stearic acid (about 70 g)
Single stearic acid glyceride (about 5 g)
White oil (about 50 ml)
Silicon oil (about 15 ml)
Octadecanol (about 5 g)
Glycerin (about 60 ml)
Snail Serum (about 150 ml)
Potassium hydroxide (about 2.5 g)
Aromatic Essence (about 5 ml)
Phenoxyethanol & Ethylhexygycerin (about 10 g)
Distilled water (about 600 ml) plus enough water to bring the weight of the product to 1000 g.

Formula 7:
Rose Hip Seed Oil (about 8 to about 10 ml)
Gastropod biological fluid filtrate (about 100 to about 150 ml)
Olive Oil emulsifier (about 8 to about 10 g)
Seaweed Extract (about 18 g to about 22 g)
Hyaluronic Acid (about 8 g to about 10 g)
Chamomile (about 28 g to about 32 g)
Saccharide Isomerate (about 38 g to about 42 g),
Licorice (Glycyrrhiza inflata) Extract (about 22 g to about 25 g)
   A preservative (about 18 g to about 22 g): made with the following botanical extracts in minute quantity: Origanum Vulgare (Oregano) Leaf Extract, Thymus Vulgaris (Thyme) Extract, Cinnamomum Zeylanicum (Cinnamon) Bark Extract, Rosmarinus Officinalis (Rosemary) Leaf Extract, Lavandula Augustifolia (Lavender) Flower Extract, Citrus Medica Limonum (Lemon) Peel Extract, Mentha Piperita (Peppermint) Leaf Extract, Hydrastis Canadensis (Golden Seal) Root Extract, and Olea Europaea (Olive) Leaf Extract
   Distilled water (about 600 ml) plus enough water to bring the weight of the product to about 1000 g.

Formula 8:
Rose Hip Seed Oil (about 10 ml)
Gastropod biological fluid filtrate (about 100 ml)
Olive Oil emulsifier (about 10 g)
Seaweed Extract (about 20 g)
Hyaluronic Acid (about 10 g)
Chamomile (about 30 g)
Saccharide Isomerate (about 40 g),
Licorice (Glycyrrhiza inflata) Extract (about 25 g)
   A preservative (about 20 g): made with the following botanical extracts in minute quantity: Origanum Vulgare (Oregano) Leaf Extract, Thymus Vulgaris (Thyme) Extract, Cinnamomum Zeylanicum (Cinnamon) Bark Extract, Rosmarinus Officinalis (Rosemary) Leaf Extract, Lavandula Augustifolia (Lavender) Flower Extract, Citrus Medica Limonum (Lemon) Peel Extract, Mentha Piperita (Peppermint) Leaf Extract, Hydrastis Canadensis (Golden Seal) Root Extract, and Olea Europaea (Olive) Leaf Extract
   Distilled water (600 ml) plus enough water to bring the weight of the product to 1000 g.

Compositions were mixed with a standard mixer until homogenous.

### Example 7

### Preparation of an Excipient Able to be Added to the Gastropod Biological Fluid to Make Cosmeceuticals

Preparation of an Excipient Preferred for Use in Therapeutic compositions of the present technology containing the gastropod biological fluid of the present technology was made by the following process: Cetyl alcohol (15 g.) was fused by standard processes and then 50 g of a mixture of polyethylene glycols [polyethylene glycol 400 (49. %), 1500 (1.0%) and 4000 (49.5%)] was added to form a first mixture. When the fusion point was reached, this mixture was transferred to a commercial pharmaceutical laboratory. A second mixture was prepared by fusing 5 g. of glycerol monostearate and adding 12.6 g (12.6 ml) of distilled water or preferably a physiological serum previously heated to 80° C. When the temperature of the second mixture was the same as the temperature of the first mixture, the second mixture was added to the first mixture. With continuous mixing, 7 g of calcium carbonate and 0.4 g of zinc oxide, both in the form of micropowders, and 10 ml of paraffin oil were added to the mixture.

### Example 8

### Preparation of Therapeutic Cream

A total of about 25 to about 30 ml of the gastropod biological fluid (or about 25 to about 30 g of the freeze-dried fluid) was mixed with about 500 to about 600 g of the excipient described above at about 30° C. to about 40° C. in a mixer. A total of about 500 to about 700 g of cream was obtained.

### Example 9

### Preparation of a Nourishing Facial Cream

A cream was prepared by mixing in a total of 200 grams, 10 ml of the gastropod biological fluid, 0.2 g of panthotenic acid, and 0.2 g of heparin in the excipient described above. The cream tones and revitalizes the skin.

### Example 10

### Preparation of a Nourishing Hand Cream

The cream was prepared by mixing about 100 g total, 5 g of the gastropod biological fluid, 4 cc. of glycerin, and 0.5 g of lanolin. The resulting cream was very smooth and it spread well. It can be applied in the day or at night to regenerate, rejuvenate, and smooth the skin.

### Example 11

### Preparation of a Clarifying and Moisturizing Liquid

This example describes the preparation of a clarifying and moisturizing liquid for dry, normal or oily skin. For use on normal and oily skin, the liquid was prepared by mixing 200 g. of the excipient, 20 cc. of the gastropod biological fluid, 0.3 g of potassium aluminum sulfate, and 10 c. of rosewater. For use on dry skin, the liquid was prepared as above except that 2 cc. of a mineral oil, e.g., paraffin oil, was added. Colorants may optionally be added. The resulting liquid cleanses and smoothes the skin and heightens the transparency of the complexion. It also serves as a base for make-up (e.g., powder). Finally, it protects the skin from the harmful action of cosmetic powders which consist of minerals which have a hardness value of 1 on the Mohr scale.

### Immediate Moisturizing

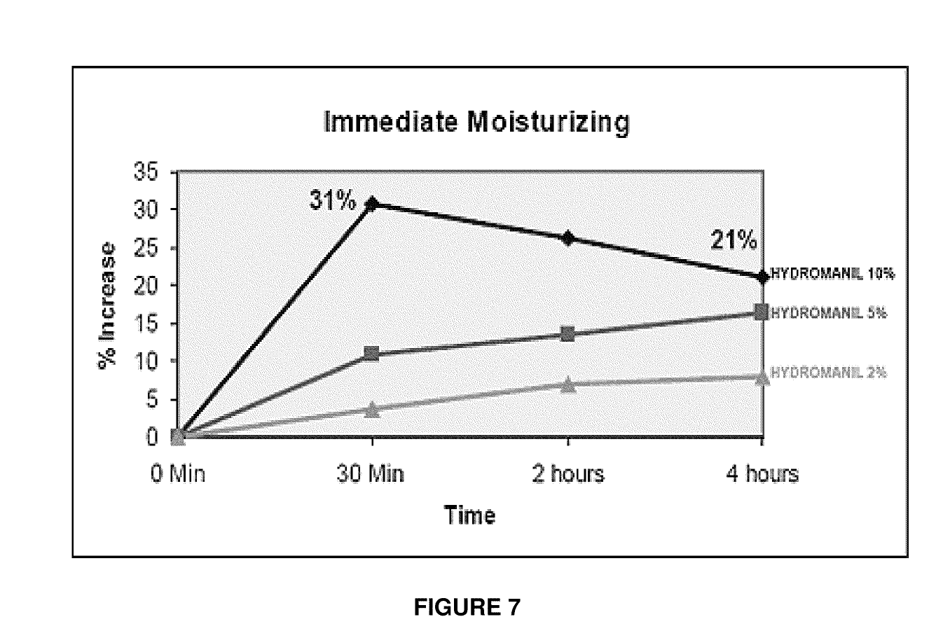

To ascertain the moisturizing effect of the gastropod biological fluid in a cream, the moisturizing kinetics of a cream containing 2%, 5% or 10% gastropod biological fluid of the present technology was measured by Corneometry. The moisture level was measured at time 0 minutes (T0), 30 minutes, 2 hours and 4 hours after a single application of the product formulated with 2%, 5% and 10% gastropod biological fluid in an aqueous gel. Results can be seen in FIG. 7. On application of the formulation with the highest dose of gastropod biological fluid (10%) was high enough to retain temporarily a part of the water contained into the cosmetic formulation. A maximum 30.8% increase in moisturizing was recorded after 30 min. Later readings (2 h, 4 h) revealed that transepidermal water accumulated without substantial dissipation. With medium and low concentrations (5% and 2%) the instantaneous effect at 30 min is not so marked, but over time still retains more moisture, reinforcing the importance of the transepidermal water retention that is believed to result from the filmogenic effect of the gastropod biological fluid by accumulating transepidermal water without dissipation (kinetic factor).

### Long-Lasting Moisturizing Effect of Cream Containing Gastropod Biological Fluid

In order to assess the long lasting moisturizing effects of creams containing the gastropod biological fluid of the present invention, the moisturizing kinetic study was extended to 21 days. The study participants applied the formulations (10% gastropod biological fluid or Placebo) twice a day to their skin. The moisture level was measured on day 22 for each study participant and the results averaged as shown in FIG. 8. The moisturizing level of the gastropod biological fluid (snail serum) on the treated area on day 22 was clearly higher (+12.5%) as compared with the initial values of each volunteer before the start of the study (D0/T0). These results demonstrate that repeated applications of this product have cumulative moisturizing effects as compared with the placebo.

### Example 12

### Preparation of a Toning and Cleansing Liquid

The liquid was prepared by mixing 100 g total, 10 cc. of the gastropod biological liquid, 100 cc. of physiological serum, 0.3 g. of potassium aluminum sulfate, 0.5 g. of boric acid, and 0.1 g. of copper sulfate. The liquid is recommended for use on delicate skin or skin where acne is present. It produces an intense refreshing feeling and a soothing, restful sensation.

### Example 13

### Preparation of an Anti-Wrinkle Cream

An anti-wrinkle cream for daily use was prepared by mixing 250 g total, 5 cc. of the gastropod biological fluid, 7 g. of lanolin, and 1.56 g. of potassium aluminum sulfate can be added to the base excipient or a physiological serum. A concentrated cream for use no more often than once a week is prepared as above except that 15 g. of the active principal, 15 g. of lanolin, and 2 g. of potassium aluminum sulfate are used. The cream is easy to apply and is used only on areas where wrinkles exist. The active ingredient is effective in reducing wrinkling, whereas the other components produce an astringent action.

Skin topography analysis for measuring the effectiveness of the anti-wrinkle cream was performed obtaining silicon imprints over a certain facial areas from selected healthy women volunteers treated with anti-wrinkle cream or placebo. Silicon imprints were obtained after 0, 15 and 30 days of twice a day treatments. To achieve a digital representation of the skin surface, a capacitive device has been used. Images are acquired in real-time by pressing the device lightly over a skin's ROI. This device, originally developed for fingerprint acquisition and recognition in biometric applications, relies on an active capacitive pixel sensing technology and it is able to capture detailed images of the skin at a resolution of 50 μm/pixel. The sensor's array is composed of 256×360 pixels and is of 12.8×18.0 mm. The surface of each pixel is composed of two adjacent metal plates. When live skin is brought near to the sensor plates, the skin interferes with field lines between the two plates and the feedback capacitance is minimized. Conversely, when the skin is moved away from the sensor surface the feedback capacitance is maximized. Therefore, the gray level values of the output image represent the 3D skin sensor gives a map of the skin surface by modulating the gray level values of each pixel in function of the capacitance measured, which is in turn a function of the distance between the sensor plate and the 3D profile of the skin surface.

It is easy to obtain detailed information from the high resolution images provided by the capacitive device. However, many factors may affect the gray level values provided by the sensor device: (1) the pressure applied to the sensor surface during acquisition; (2) the hydrating state of the skin locally affects the gray level values of the resulting capacitive image; (3) the output response of the capacitive device varies according to pixel displacements, as the sensor shows a different behavior in the upper part of the cell's array; (4) the capacitive background image (the image acquired without anything on top of the device sensor area, hereinafter briefly background) tends to deflect to higher gray level values; and (5) noise alters the final gray level values of a capacitive image. Facing these problems is crucial to infer real absolute metric measures from the skin topographic structures as detected in the capacitive images.

The first part of experiments is to measure planar distances between well identified topographic structures. We paid our attention on wrinkles, identified by high gray level values with respect to the surrounding skin, which in contrast appear darker in the capacitive images. First, a preprocessing step is performed with the aim of reducing misleading factors described above. Then we extract wrinkle's profiles which are transformed in an analytical form through using natural cubic spline. After that, what we measure is the inter-wrinkle distance (hereinafter, briefly/WD), that is the distance, in micron, between axes passing by two following local minima extracted from the 2D profile and belonging to adjacent wrinkles. The second part of the experiments is devoted to validate these measures by comparing them with measures of the same body site area achieved through profilometric analysis. Silicone skin replicas have been analyzed with an optical profilometer equipped with a 20× magnification objective and a 0.5× scale factor reducer lens, thus giving an overall magnification factor of 10×. The system, based on white light interferometry, provides accurate surface data depths of a limited region of the replica, being the field of view of 0.62×0.47 mm2 and 640×480 pixel the image resolution.

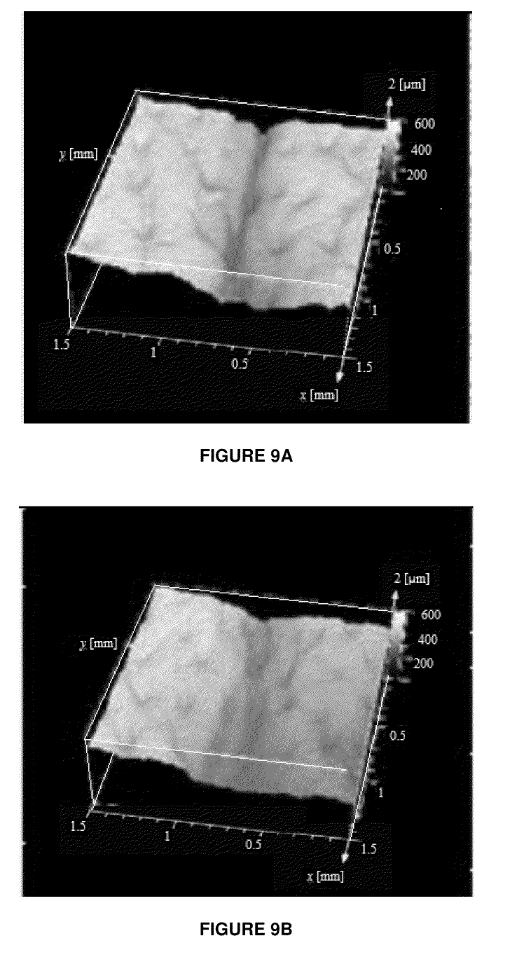

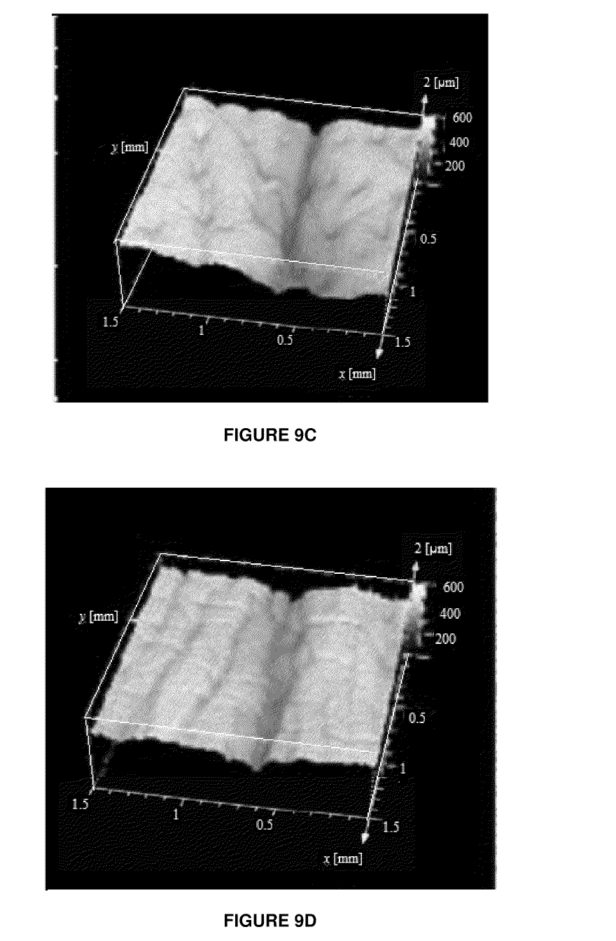

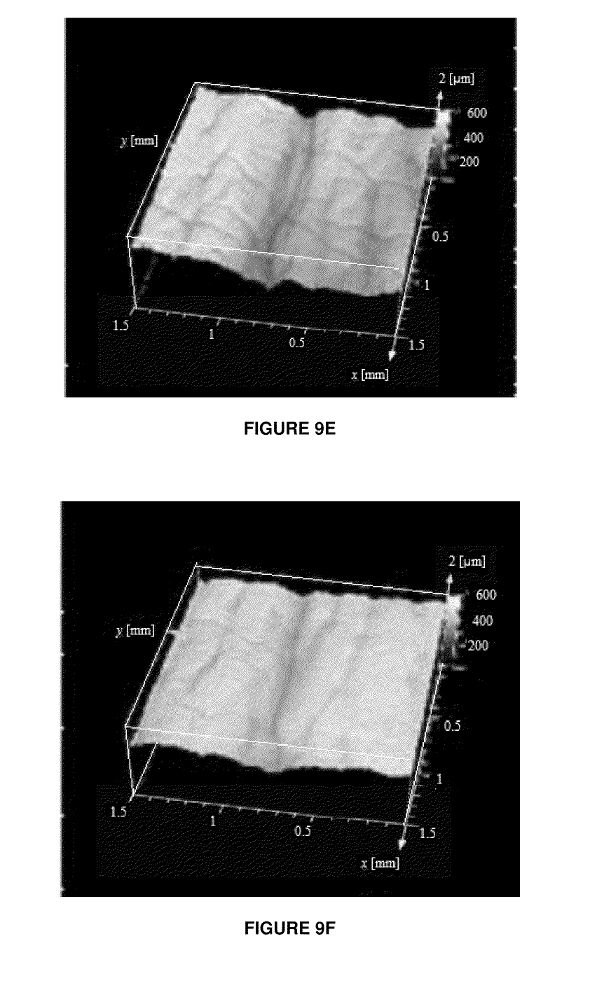

The concentrated cream was tested on more than 200 healthy woman volunteers. The group was divided and tested with anti-wrinkle cream describe above or placebo for 30 days. The anti-wrinkle effect lasted for approximately one week after each application. The more numerous and larger the wrinkles, the greater was the visible effect of the treatment via follow-up observational analysis. As shown by a representative result illustrated in FIG. 9, the depth of wrinkles was diminished after 15 and 30 days for skin treated with the anti-wrinkle cream (FIGS. 9E and F) as compared with placebo (FIGS. 9B and C). This observation that the depth of the wrinkle is significantly decreased after 30 days of treatment confirms the anti-wrinkle benefits of cream containing the gastropod biological fluid of the present technology.

### Example 14

### Preparation of a Sunscreen Creams

A sunscreen cream was prepared by mixing 250 g total with 5 ml of the gastropod biological fluid filtrate. A sunscreen tanning cream is prepared as above except that 1 g of metallic iodine and 0.3 g of Vitamin C are added. As a preventative, one thin application of the sunscreen before exposure to the sun is sufficient. If one has suffered sunburn, a thin coating of the cream may be applied every six hours.

To test the sunscreen, the sunscreen cream was used in the treatment of 306 fair-complexioned test subjects. The products were used preventively in 6% of the cases and curatively in 94% of the cases. The sunscreen was used in treating test subjects who suffered sunburns at swimming pools with highly chlorinated water, where the subjects showed blistering (phlytena), and persons who suffered sunburns at beaches. In every case, the cream eliminated the pain and burning sensation within seconds of being applied, blisters disappeared in 5 to 6 hours; and the sunburn was gone in 24 hours.

Ultraviolet irradiation causes adverse effects like sunburn, photosensitivity reactions or immunologic suppression. The aim of this study was to evaluate the photo-protective outcome of Pharmacom™ Younger Sunscreen Cream by the determination of erythema indexes and the assessment of the high molecular weight antioxidants and its metabolites in human dermis. These substances were used as markers of oxidative effect. Eight healthy female subjects were enrolled in this study. Two abdominal areas were exposed to solar simulated irradiation with three minimal erythema doses, one with the Sunscreen Cream application and the other site without the Sunscreen Cream application. Two other areas were used as control, one without SPF8 application and the other site after SPF8 application. Ascorbic acid and its metabolites (dehydroascorbic acid, threonic acid, oxalic acid and xylose) were collected from human dermis by microdialysis and assessed by gas chromatography mass spectrometry. Irradiated site without sunscreen application had significantly demonstrated lower dermis ascorbic acid concentrations and a higher erythema index than the three other sites (P<0.05). Threonic acid, oxalic acid and xylose dermis concentrations were significantly higher in site III than in the control site I (P<0.05). The protected-irradiated site did not show erythema formation and there was stability of ascorbic acid dermis concentrations with non-variation in its metabolites. These results suggest that sunscreen compositions of the present technology may act as a sunscreen and prevent oxidative and UV damage to the skin.

The present technology is now described in such full, clear and concise terms as to enable a person skilled in the art to which it pertains, to practice the same. It is to be understood that the foregoing describes preferred embodiments of the present technology and that modifications may be make therein without departing from the spirit or scope of the present technology as set forth in the appended claims.

1. A method of collecting a gastropod biological fluid comprising the steps of:
1) stimulating a live gastropod to increase biological fluid secretion;
2) collecting the secreted biological fluid;
3) centrifuging the secreted fluid to create a supernatant; and
4) filtering the supernatant to recover a filtrate containing an enhanced amount of glycosaminoglycan.

2. The method of claim 1, further comprising the step of fasting the gastropod for about 1 day to about 5 days to reduce toxins in the secreted biological fluid.

3. The method of claim 1, wherein the gastropod is a Helix Aspersa Müller.

4. The method of claim 1, wherein the gastropod is Helix pomatia.

5. The method of claim 1, wherein the gastropod biological fluid comprises (1→→4)-2-acetyl,2-deoxy-α-D-glucopyranose (1→4)-2-sulfo-α-L-idopyranosyluronic acid.

6. The method of claim 1, wherein the step of stimulating the live gastropod is a method selected from the group comprising: exposing the gastropod to sound vibrations; exposing the gastropod to low hyperbaric pressure; exposing the gastropod to hypoxic conditions; exposing the gastropod to a thermal puncture; exposing the gastropod to ionizing radiation; exposing the gastropod to a physical stimulus; and inverting the gastropod.

6. The method of claim 5, wherein the step of exposing the gastropod to sound vibration comprises exposing the gastropod to ultrasonic radiation of about 20 kHz for at least about 10 seconds.

7. The method of claim 5, wherein the step of exposing the gastropod to low hyperbaric pressure comprises exposing the gastropod to a hyperbolic chamber with a pressure of about 4 psi to about 7.5 psi for at least about 10 seconds.

8. The method of claim 7, wherein the gastropod is exposed to the hyperbaric chamber for at least about 15 seconds.

9. The method of claim 5, wherein the steps of exposing the gastropod to a physical stimulus comprises centrifugation of the gastropod between about 1 G to about 6 G force for about 2 minutes to about 10 minutes.

10. The method of claim 9, wherein the centrifugation step is performed at a temperature of about 10° C. or greater.

11. The method of claim 9, wherein the centrifugation step is performed at a temperature of about 15° C. or greater.

12. The method of claim 9, wherein the centrifugation step is performed at a temperature of about 20° C. or greater.

13. The method of claim 9, wherein the centrifugation step is performed at a temperature of between about 20° C. to about 35° C.

13. A gastropod biological fluid made by the method of claim 1.

14. The gastropod biological fluid of claim 13, wherein the gastropod biological fluid contains an effective amount of the glycosaminoglycan to heal human or animal skin tissue.

15. A cosmeceutical that promotes skin rejuvenation and healing comprising at least about 15% by weight of the filtrate made by the method of claim 1.

16. The cosmeceutical of claim 15, further comprising a delivery system.

17. The cosmeceutical of claim 16, wherein the delivery vehicle comprises a cream, a gel, a solution, an oil, a suspension, a dispersion, an ointment, a membrane, a patch, a mask, a pill, a suppository, a soap, a skin adhesive glue, a powder, a derivative thereof, or a combination thereof.

18. A gastropod biological fluid comprising:
about 0.001% to about 0.1% uronic acid;
about 0.003% to about 0.3% hexozamine;
about 0.001 to about 0.1% acetyl;
about 0.001 to about 0.1% sulfate;
about 0.02% to about 5.0% of at least one mucopolysaccharide
about 0.05% to about 0.5% of at least one fibroblast growth factor;
about 0.01% to about 0.1% of at least one enzyme;
about 0.01% to about 1.0% hyaluronic acid;
about 0.001% to about 0.1% of at least one oxygen carrier;
about 0.001% to about 0.1% of at least one high molecular weight protein;
about 0.0001 to about 0.01% of at least one trace element;
about 0.005% to about 0.1% of at least one high molecular weight antioxidant compound;
about 0.005% to about 0.1% of at least one low molecular weight compound; and
about 80% to about 98% water.

19. A gastropod biological fluid comprising:
about 0.02% to about 0.03% uronic acid;
about 0.03% to about 0.05% hexozamine;
about 0.01 to about 0.02% acetyl;
about 0.01 to about 0.02% sulfate;
about 0.28% to about 0.30% of at least one mucopolysaccharide
about 0.1% to about 0.2% of at least one fibroblast growth factor;
about 0.05% to about 0.06% of at least one enzyme;
about 0.1% to about 0.2% hyaluronic acid;
about 0.01% to about 0.02% of at least one oxygen carrier;
about 0.04% to about 0.05% of at least one high molecular weight protein;
about 0.001 to about 0.002% of at least one trace element;
about 0.05% to about 0.06% of at least one high molecular weight antioxidant compound;
about 0.05% to about 0.06% of at least one low molecular weight compound; and
about 96.0% to about 96.5% water.

20. The gastropod biological fluid of claim 19, wherein the at least one mucopolysaccharide comprises (1→4)-2-acetyl,2-deoxy-α-D-glucopyranose (1→4)-2-sulfo-α-L-idopyranosyluronic acid.

21. The gastropod biological fluid of claim 19, wherein the at least one fibroblast growth factor is a member selected from the group comprising FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, and a combination thereof.

22. The gastropod biological fluid of claim 19, wherein the at least one fibroblast growth factor is FGF1, FGF4, FGF7, FGF10, or a combination thereof.

23. The gastropod biological fluid of claim 19, wherein the at least one enzyme comprises collegenase, gelatinase, proteoglycanas, a derivative thereof, or a combination thereof.

24. A cosmeceutical comprising at least about 1% to about 30% by weight of a gastropod biological fluid containing (1→4)-2-acetyl,2-deoxy-α-D-glucopyranose (1→4)-2-sulfo-α-L-idopyranosyluronic acid.

25. The cosmeceutical of claim 24, comprising at least about 10% to about 25% by weight of the gastropod biological fluid.

26. The cosmeceutical of claim 24, wherein the gastropod biological fluid is a supernatant biological fluid.

27. The cosmeceutical of claim 24, where the gastropod biological fluid is a filtrate containing an effective amount of (1→4)-2-acetyl,2-deoxy-α-D-glucopyranose (1→4)-2-sulfo-α-L-idopyranosyluronic acid.

28. The cosmeceutical of claim 24, further comprising an effective amount of squalene.

29. The cosmeceutical of claim 28, wherein the squalene comprises about 0.3% to about 3.0% by weight of the cosmeceutical.

30. The cosmeceutical of claim 29, wherein the squalene comprises about 0.8% to about 1.0% by weight of the cosmeceutical.

31. The cosmeceutical of claim 24, further comprising an effective amount of hyaluronic acid.

32. The cosmeceutical of claim 31, wherein the hyaluronic acid comprises about 0.3% to about 3.0% by weight of the cosmeceutical.

33. The cosmeceutical of claim 31, wherein the hyaluronic acid comprises about 0.5% to about 1.2% by weight the cosmeceutical.

34. A skin care composition comprising:
a biologically effective amount of a gastropod excreted fluid, and
a delivery vehicle capable to be applied to human or animal skin.

35. The skin care composition of claim 34, wherein the delivery vehicle comprises a cream, a gel, a solution, an oil, a suspension, a dispersion, an ointment, a membrane, a patch, a mask, a pill, a suppository, a soap, a skin adhesive glue, a powder, a derivative thereof, or a combination thereof.

36. The skin care composition of claim 34, further comprising about 0.3% to about 3.0% squalene and about 0.3% to about 3.0% hyaluronic acid.

37. The skin care composition of claim 32, further comprising at least one preservative, at least one antioxidant, at least one solvent, at least one whitening agent, and at least one abrasive agent.

38. The skin care composition of claim 37, wherein the at least one preservative comprises phenoxyethanol, ethylhexylglycerin or a combination thereof.

39. The skin care composition of claim 37, wherein the at least one preservative comprises about 0.1% to about 4.0% by weight of the skin care composition.

40. The skin care composition of claim 37, wherein the antioxidant is a member selected from the group consisting of olive derivatives, oleic acid, palmitic acid, uric acid, carotenoids, ubichinones, lipoic acid, vitamin C, vitamin E, phenolic compounds, resveratrol, beta carotene, selenium, high molecular weight antioxidant compounds, low molecular weight antioxidant compounds, derivatives thereof and combinations thereof.

41. The skin care composition of claim 37, wherein the antioxidant is about 0.1% to about 10% by weight of the total weight of the skin care composition.

42. The skin care composition of claim 37, wherein the antioxidant is about 2.0% to about 3.0% by weight of the total weight of the skin care composition.

43. The skin care composition of claim 37, wherein the solvent is a member selected from the group consisting of glycerin, water, or a combination thereof.

44. The skin care composition of claim 43, wherein the solvent comprises about 60.0% to about 70.0% water and about 10.0% to about 20.0% glycerin based on the total weight of the skin care composition.

45. The skin care composition of claim 37, wherein the solvent is about 50.0% to about 90.0% of the total weight of the skin care composition.

46. The skin care composition of claim 37, wherein the abrasive agent is corundum oxide.

47. The skin care composition of claim 37, wherein the abrasive agent comprises about 0.001% to about 0.005% of the total weight of the skin care composition.

48. The skin care composition of claim 34, further comprising an olive oil derivative.

49. The skin care composition of claim 48, wherein the olive oil derivative is selected from the group consisting of sorbitan olivate, Peg 4 olivate, squalene, a derivative thereof, and a combination thereof.

50. The skin care composition of claim 48, wherein the olive oil derivative comprises about 0.5% to about 5.0% of the total weight of the skin care composition.

51. The skin care composition of claim 34, further comprising about 2.0% to about 3.0% by weight of an olive oil derivative, about 10% to about 20% by weight of the gastropod biological fluid, about 0.5% to about 2.0% by weight of hyaluronic acid, about 10% to about 15% by weight of glycerin, about 0.5 to 1.5% by weight of a preservative comprising phenoxyethanol and ethylhexygycerin, and about 0.1 to about 1.0% corundum oxide by weight of the total weight skin care composition.

52. A composition comprising:
about 10.0% to about to about 25.0% of a gastropod excreted fluid;
about 0.5% to about 10.0% stearic acid;
about 0.1% to about 1.0% single stearic acid glyceride;
about 1.0% to about 10.0% of at least one oil;
about 0.1% to about 1.0% octadecanol;
about 1.0% to about 10.0% glycerin;
about 0.02% to about 2.0% potassium hydroxide; and
about 0.2% to about 2.0% of a preservative.

53. The composition of claim 52, wherein the at least one oil comprises white oil, silicon oil, or a combination thereof.

54. The composition of claim 53, wherein the at least one oil comprises about 1% to about 6% white oil and about 1% to about 2% silicon oil.

55. The composition of claim 52, further composing about 0.1% to about 1.0% of an aromatic essence.

56. A formulation comprising:
about 10% to about to about 20% of a gastropod excreted fluid;
about 0.2% to about to about 2.0% rose hip seed oil;
about 0.2% to about 2.0% of a olive oil emulsifier;
about 0.5% to about 5.0% of a seaweed extract;
about 0.1% to about 1.0% of hyaluronic acid;
about 0.4% to about 4.0% chamomile extract;
about 0.5% to about 5.0% saccharide isomerate;
about 0.3% to about 3.0% licorice extract; and
about 0.3% to about 3.0% of a preservative.

57. The formulation of claim 56, further comprising about 60% to about 65% water.

58. The formulation of claim 56, wherein the preservative comprises one or more botanical extracts.

59. The formulation of claim 58, wherein the botanical extracts are a member selected from the group consisting of organum vulgare leaf extract, thymus vulgaris extract, cinnamomum zeylanicum bark extract, rosmarinus officinalis leaf extract, lavandula augustifolia flower extract, citrus medica limonum peel extract, menthe piperita leaf extract, hydrastis Canadensis root extract, olea europaea leaf extract, derivatives thereof, and a combination thereof.
